(19) United States
(12) Patent Application Publication
(10) Pub. No.: US 2007/0155701 A1
(43) Pub. Date: Jul. 5, 2007

(54) KETO CANNABINOIDS WITH THERAPEUTIC INDICATIONS

(76) Inventors: Alexandros Makriyannis, Watertown, MA (US); Dai Lu, Melrose, MA (US)

Correspondence Address: ALIX YALE & RISTAS LLP, 750 MAIN STREET, SUITE 1400, HARTFORD, CT, 06103, US

(21) Appl. No.: 11/619,931

(22) Filed: Jan. 4, 2007

Related U.S. Application Data
(63) Continuation-in-part of application No. 10/647,544, filed on Aug. 25, 2003, now Pat. No. 7,183,313.
(60) Provisional application No. 60/405,940, filed on Aug. 26, 2002.
(60) Provisional application No. 60/405,608, filed on Aug. 23, 2002.

(51) Int. Cl. A61K 31/675; A61K 31/541; A61K 31/5377; A61K 31/496; A61K 31/473; A61K 31/382; A61K 31/353; C07D 417/02; C07D 413/02; C07D 409/02; C07D 405/02

(52) U.S. Cl. 514/80; 514/227.8; 514/232.8; 514/298; 514/455; 514/437; 544/60; 546/108; 544/126; 544/146; 544/151; 544/361; 546/22; 549/26; 549/290; 514/252.13; 514/253.03; 514/254.11

(57) ABSTRACT

Novel tricyclic cannabinoid compounds are presented. Some of these compounds exhibit fluorescence properties. The fluorescent cannabinoid compounds are typically endogenously fluorescent. Some of these compounds, when administered in a therapeutically effective amount to an individual or animal, result in a sufficiently high level of that compound in the individual or animal to cause a physiological response. The physiological response useful to treat a number of physiological conditions.

### CROSS REFERENCE TO RELATED APPLICATIONS

This application is a division of U.S. application Ser. No. 10/647,544 filed Aug. 25, 2003 which claims the benefit of United States Provisional Application No. 60/405,608, filed Aug. 23, 2002 and United States Provisional Application No. 60/405,940, filed Aug. 26, 2002, the contents of each of which are incorporated by reference in their entirety.

### STATEMENT REGARDING FEDERALLY SPONSORED RESEARCH OR DEVELOPMENT

The disclosed work may have been done with U.S. Government support under Contract No. DA3801 and DA7215 awarded by the National Institute of Health. The U.S. Government has certain rights in the invention.

### FIELD

This disclosure relates generally to cannabinoid compounds. One embodiment of the present disclosure more particularly relates to cannabinoid compounds exhibiting fluorescence properties, particularly in the ultraviolet-visible wavelength ranges.

### BACKGROUND

The classical cannabinoid Δ⁹-Tetrahydrocannabinol (Δ⁹-THC) is the major active constituent extracted from Cannabis sativa. The effects of this, and other, cannabinoids are due to an interaction with specific, high-affinity receptors. Presently, two cannabinoid receptors have been characterized: CB1 and CB2.

Characterization of these receptors has been made possible by the development of specific synthetic ligands such as the agonists WIN 55212-2 and CP 55,940.

Additionally, recent scientific discoveries have demonstrated that the endocannabinoid system is very extensive and is currently under intense investigation. Radiochemical methods have been in use for more than a decade for studying the complex phenomena associated with the endocannabinoid system and cannabimimetic molecules. Despite the usefulness and sensitivity of radiochemical methods, the use of alternative methods such as fluorescence techniques can provide information not readily accessible by conventional radiochemical methods and circumvent certain drawbacks associated with them, such as high cost, special precautions in handling and disposal and potential health hazards. Fluorescent approaches provide great advantages over radiochemical methods in accuracy, sensitivity, efficiency, safety and a wide scope of additional applications, and generally are less costly than radiochemical methods. The state-of-art fluorescence approaches enable researchers to detect particular components of complex biomolecular assemblies, including living cells. In particular the emission spectrum of a fluorescer is sensitive to its environment. Therefore, fluorescence approaches are extremely useful in providing spatial, dynamic and temporal information about the interactions between macromolecules and their ligands.

With the help of available fluorescent ligands, fluorescence techniques have successfully been applied to study the behavior of a number of biological macromolecules, including dopamine receptors, histamine receptors, muscarinic receptors, adrenergic receptors, glucagon receptors, opiate receptors, adenosine receptors and serotonin receptors. The applications of receptor-specific fluorescent ligands are considerably broad, such as molecular studies on ligand-induced conformational changes within the receptor, rapid kinetics of ligand-receptor interactions, the localization of the ligand-binding site on the receptor and distances between different binding sites on the same receptor. Moreover, fluorescent ligands have been successfully used for studying the mobility of some receptors in both normal and pathophysiological conditions by fluorescence photobleach recovery techniques, and to localize receptors at tissue and cellular level by fluorescence microscopic techniques. Furthermore, receptor-specific fluorescent ligands have been employed for receptor assays including the determination of the receptor dissociation constant (K_{D}) and the total receptor content of the tissue (Bₘₐₓ) by fluorescence titration techniques.

In general, fluorescent ligands are prepared by linking parent ligands with fluorescent moieties to make the newly formed ligands detectable or measurable by fluorescence techniques. Such strategies often face the challenge of reduced potency or efficacy of the parent ligands during interaction with target macromolecules. The inventors are not aware of cannabinoid compounds having fluorescence properties.

### SUMMARY

One aspect of the present disclosure comprises compounds exhibiting fluorescent properties. The fluorescent compounds described in compound formulas I and II are typically endogenously fluorescent and do not rely on linking the cannabinoid compound to a fluorescent moiety. At present, the inventors believe that the compounds described in the compound formulas will have fluorescent properties as long as a long conjugation system (typically comprising the phenyl A ring and a double bond as well as a carbonyl group) can be formed within the tricyclic cannabinoid structure and the Y moiety comprises an electron rich structural element such as nitrogen and oxygen. Some fluorescent cannabinoids not only are capable of generating strong fluorescence, but also can act as high affinity modulators for cannabinoid receptors, and are therefore, of potential usefulness as therapeutic agents through the modulation of the CB1 and/or CB2 cannabinoid receptors.

Another aspect of the present disclosure comprises compounds exhibiting cannabimimetic activity, both fluorescent and non-fluorescent, pharmaceutical preparations employing these compounds and methods of administering therapeutically effective amounts of these compounds to provide a physiological effect.

Yet another aspect of the disclosure comprises methods of utilizing the fluorescent compounds.

The novel fluorescent cannabinoid compounds exhibit strong fluorescence, for example in the ultraviolet-visible wavelength ranges. The emission wavelength of some of the disclosed compounds ranges from about 390 nm to about 550 nm. The molar extinction constants for some of the disclosed compounds ranges from about 1.5×10⁴ to about 2.34×10⁴ (1/Molxcm/L).

Surprisingly, the cannabinoid compounds in one aspect of the disclosure, comprising the A, B and C rings, are intrinsically fluorescent and do not rely on linkage with a fluorescent moiety to achieve their fluorescent properties. Since these fluorescent cannabinoid compounds are intrinsically fluorescent, problems with reduced potency or efficacy are avoided.

In some embodiments the disclosed fluorescent cannabinoids not only are capable of generating strong fluorescence, but also can act as high affinity modulators for cannabinoid receptors, and are therefore, of potential usefulness as therapeutic agents through the modulation of the CB1 and/or CB2 cannabinoid receptors.

In general, the compositions of the disclosure may be alternately formulated to comprise, consist of, or consist essentially of, any appropriate components herein disclosed. The compositions of the disclosure may additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

One embodiment of the disclosure may be represented by compound formula I, and physiologically acceptable salts thereof, 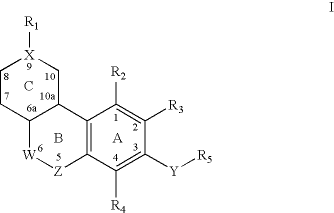
wherein:

the C ring contains one double bond.

W comprises C═O, C═S or C═CH₂. Compound formula I will have advantageous fluorescence properties when W is C═O and the C ring has a double bond in the 6a-10a position. It is believed that compound formula I will have advantageous fluorescence properties when R1 is ═O and the C ring has a double bond in the 10-10a position.

X comprises C, CH, N, S, O, SO or SO₂.

Y comprises O, S, NH, N-alkyl, N═N, C═C or C≡C.

Z comprises O, NH, N-alkyl where the alkyl group has 1 to about 5 carbon atoms or N-substituted alkyl, where the alkyl group has 1 to about 5 carbon atoms and is substituted with at least one substituent group in any possible position.

When X is S, O, SO or SO₂, R₁ is not present.

When X is N, R₁ comprises H, alkyl, alkoxy-alkyl, alkylmercapto, alkylamino, SO₃alkyl, SO₂NQ₁Q₂, CONQ₁Q₂, COC(halogen)₃ or alkyl substituted in any possible position with at least one member selected from OH, CHO, COOH, C(halogen)₃, N₃, NCS, CN, PO₃H₂, SO₃H, or SO₃alkyl.

When X is C or CH, R₁ comprises any possible member selected from H, halogen, N₃, NCS, CN, NO₂, NQ₁Q₂, ═O, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, ═CH₂, alkyl, alcohol, alkoxy, alkylmercapto, alkylamino, di-alkylamino or alkyl substituted in any possible position with at least one substituent group as later defined.
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
   Q₃ comprises H, alkyl, hydroxyloweralkyl, or alkyl-NQ₁Q₂.

In one advantageous variation X is C or CH and R₁ comprises any possible member selected from H, halogen, ═CH₂, an alkyl group having 1 to about 5 carbon atoms or an alkyl group having 1 to about 5 carbon atoms and substituted in any possible position with at least one member selected from OH, CHO, COOH, CH₂OH, halogen, C(halogen)₃, N₃, NCS, CN, PO₃H₂, SO₃H, or SO₃alkyl, SO₂NQ₁Q₂, CONQ₁Q₂, COC(halogen)₃, NQ₁Q₂.

R₂ comprises H, OH, OCH₃, OPO₃H₂, OSO₃H, PO₃H₂, SO₃H, halogen, NQ₁Q₂, COOQ₃, OQ₃, CQ₃, C(halogen)₃, alkyl-hydroxyl, NH—COalkyl, NH—COaryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂.
T₁ is in any possible position and comprises PO₃H, SO₃H, an alkyl group containing from 1 to about 16 carbons, tetrahydropyrrole, morpholine, thiomorpholine, piperazine, a heterocyclic ring or NQ₁Q₂;
   T₁ may be substituted in any possible position with at least one member selected from a substituent group, OPO₃H₂, OSO₃H, PO₃H₂, a heterocyclic ring or a heteroaromatic ring;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
Q₃ comprises H, alkyl, hydroxyloweralkyl, or alkyl-NQ₁Q₂.

R₃ comprises H, OH, halogen, C(halogen)₃, CN, N₃, NCS, NQ₁Q₂ or an alkyl group having 1 to about 4 carbon atoms,
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₄ comprises H, OH, halogen, CN, N₃, NCS, NQ₁Q₂ or an alkyl group having 1 to about 4 carbon atoms;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₅ comprises -D₁-D₂-T₂ or -D₂-T₂,
D₁, is optionally present and if present, comprises an alkyl group, a carbocyclic ring, a heterocyclic ring, N-alkyl or NH,
D₂ comprises an alkyl group having from one to about sixteen carbon atoms, CH═HC, C═C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃ or adamantan-2-ylidenemethyl-T₃, alkylamino, di-alkylamino or NH
T₂ comprises, in any possible position, a substituent group as later defined or —CO-T₄,
T₃ comprises an alkyl group having from 0 to about 9 carbon atoms,
T₄ comprises H, C-(halogen)₃, OH, NH₂, alkylamino, di-alkylamino, NO₂, alkyl, alkoxy, a heterocyclic ring or a heteroaromatic ring.

In one advantageous variation R₅ comprises -D₁-D₂-T₂ or -D₂-T₂,

D₁ is optionally present and if present, comprises an alkyl, a carbocyclic ring having 5 to 6 ring members, a heterocyclic ring having 5 to 6 ring members and 1,3 di-heteroatoms each independently selected from O, S, N and NH,
D₂ comprises an alkyl group having from one to about sixteen carbon atoms, CH═CH, C≡C, a bicyclic ring, a tricyclic terpine, 1-adamantyl-T₃, 2-adamantyl -T₃, adamantan-1-ylmethyl-T₃ or adamantan-2-ylidenemethyl-T₃, alkylamino, di-alkylamino or NH
T₂ comprises, in any possible position, a substituent group as later defined or —CO-T₄,

T₃ comprises an alkyl group having from 0 to about 9 carbon atoms,

T₄ comprises alkyl, a heterocyclic ring or a heteroaromatic ring.

Another embodiment of the disclosure may be represented by compound formula II, and physiologically acceptable salts thereof, 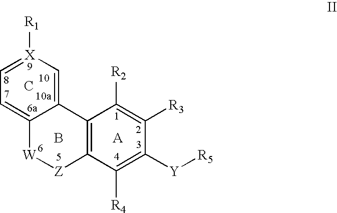
wherein:

W comprises C═O, C═S, or C═CH₂. Advantageously, W comprises C═O. Compound formula II will have advantageous fluorescence properties when W is C═O. It is believed that compound formula 11 will have advantageous fluorescence properties when R1 is ═O.

X comprises C, CH or N.

Y comprises O, S, NH, N-alkyl, N═N, C═C or C≡C.

Z comprises O, NH, N-alkyl where the alkyl group has 1 to about 5 carbon atoms or N-substituted alkyl, where the alkyl group has 1 to about 5 carbon atoms and is substituted with at least one substituent group in any possible position.

R₁ comprises any possible member selected from H, halogen, N₃, NCS, CN, NO₂, NQ₁Q₂, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, alkyl, alkyl substituted in any possible position with at least one substituent group as later defined.
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
Q₃ comprises H, alkyl, alcohol, or alkyl-NQ₁Q₂.

In one advantageous variation R₁ comprises any possible member selected from H, halogen, OH, an alkyl group having 1 to about 5 carbon atoms or an alkyl group having 1 to about 5 carbon atoms and substituted in any possible position with at least one member selected from OH, CHO, COOH, C(halogen)₃, COC(halogen)₃, N₃, NCS, CN, PO₃H₂, SO₃H, or SO₃alkyl.
R₂ comprises H, OH, OCH₃, OPO₃H₂, OSO₃H, PO₃H₂, SO₃H, halogen, C(halogen)₃, alcohol, NQ₁Q₂, COOQ₃, OQ₃, alkyl-hydroxyl, NH—COalkyl, NH—COaryl, O—COalkyl, O—COalkyl-T₁, O—CO-T₁, SO₂NQ₁Q₂, CONQ₁Q₂, NH—COalkyl-T₁, NH—CO-T₁, O-alkyl-T₁, O-T₁, NH-alkyl-T₁, NH-T₁, SO₃alkyl, SO₂NQ₁Q₂.
   T₁ is in any possible position and comprises PO₃H, SO₃H, an alkyl group containing from 1 to about 16 carbon atoms, tetrahydropyrrole, morpholine, thiomorpholine, piperazine, a heterocyclic ring or NQ₁Q₂;
      T₁ may be substituted in any possible position with at least one member selected from a substituent group, OPO₃H₂, OSO₃H, PO₃H₂, a heterocyclic ring or a heteroaromatic ring;
   Q₁ and Q₂ each independently comprise H or alkyl, or
   Q₁ and Q₂ together comprise part of a heterocyclic ring having about 4 to about 7 ring members and optionally one additional heteroatom selected from O, N or S, or
   Q₁ and Q₂ together comprise part of an imide ring having about 5 to about 6 members.
   Q₃ comprises H, alkyl, alcohol, or alkyl-NQ₁Q₂.

R₃ comprises H, OH, halogen, C(halogen)₃, CN, N₃, NCS, NQ₁Q₂ or C1 to C4 alkyl,
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₄ comprises H, OH, halogen, C(halogen)₃, CN, N₃, NCS, NQ₁Q₂ or C1 to C4 alkyl;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₅ comprises -D₁-D₂-T₂ or -D₂-T₂,
D₁ is optionally present and if present, comprises an alkyl, a carbocyclic ring, a heterocyclic ring, alkylamino, di-alkylamino or NH,
D₂ comprises an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃, or adamantan-2-ylidenemethyl-T₃, alkylamino, di-alkylamino or NH,
T₂ comprises, in any possible position, a substituent group as later defined or —CO-T₄,
T₃ comprises an alkyl group having from 0 to about 9 carbon atoms,
T₄ comprises H, C(halogen)₃, OH, NH₂, NO₂, alkyl, alkoxy, a heterocyclic ring or a heteroaromatic ring.

In one advantageous variation R₅ comprises -D₁-D₂-T₂ or -D₂-T₂

D₁ comprises alkylamino, di-alkylamino, NH, a carbocyclic ring having 4 to 6 ring members or a heterocyclic ring having 4 to 6 ring members and 1,3 di-heteroatoms each heteroatom independently selected from O, S and N,

D₂ comprises an alkyl group having from one to about sixteen carbon atoms, CH═CH, C≡C, a bicyclic ring, a tricyclic terpine, 1-adamantyl-T₃, 2-adamantyl-T₃, adamantan-1-ylmethyl-T₃ or adamantan-2-ylidenemethyl-T₃, alkylamino, di-alkylamino or NH
T₂ comprises, in any possible position, a substituent group as later defined or —CO-T₄,

T₃ comprises an alkyl group having from 0 to about 9 carbon atoms,

T₄ comprises alkyl, a heterocyclic ring or a heteroaromatic ring.

Another embodiment of the disclosure may be represented by either compound formula I or II, wherein:

The C ring in compound formula I contains a double bond in the 6a-10a position.

W is C═O.

X comprises C or N.

Y comprises O, S, NH, N-alkyl, N═N, C═C, C≡C,

Z is O.

R₁ comprises OH, CH₂OH, in compound formula I; or methyl, OH, CH₂OH in compound formula II.

R₂ comprises H, OH, OCH₃, OPO₃H₂, OSO₃H, PO₃H₂, SO₃H, halogen, C(halogen)₃, alcohol, NQ₁Q₂, COOQ₃, OQ3, NH—COalkyl, NH—CO-aryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂
T₁ is in any possible position and comprises PO₃H, SO₃H, an alkyl group containing from 1 to about 16 carbon atoms, tetrahydropyrrole, morpholine, thiomorpholine, piperazine, a heterocyclic ring or NQ₁Q₂;
T₁ may be substituted in any possible position with at least one member selected from a substituent group, OPO₃H₂, OSO₃H, PO₃H₂, a heterocyclic ring or a heteroaromatic ring;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
Q₃ comprises H, alkyl, hydroxyloweralkyl, or alkyl-NQ₁Q₂.

R₃ comprises H, OH, halogen, C(halogen)₃, CN, N₃, NCS, NQ₁Q₂ or an alkyl group having 1 to about 4 carbon atoms,
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₄ comprises H, OH, halogen, C(halogen)₃, CN, N₃, NCS, NQ₁Q₂ or an alkyl group having 1 to about 4 carbon atoms;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₅ comprises -D₁-D₂-T₂ or -D₂-T₂,
D₁ is optionally present and if present, comprises an alkyl, a carbocyclic ring, a heterocyclic ring, alkylamino or NH.
D₂ comprises an alkyl group having from one to about sixteen carbon atoms, CH═CH, C≡C, a bicyclic ring, a tricyclic ring, 1-adamantyl-T₃, 2-adamantyl -T₃, adamantan-1-ylmethyl-T₃, or adamantan-2-ylidenemethyl-T₃, alkylamino, di-alkylamino or NH.
T₂ comprises, in any possible position, a substituent group as later defined or —CO-T₄,
T₃ comprises an alkyl group having from 0 to about 9 carbon atoms,
T₄ comprises H, C(halogen)₃, OH, NH₂, NO₂, alkyl, alkoxy, alkylamino, di-alkylamino, a heterocyclic ring or a heteroaromatic ring.

In one advantageous variation R₅ comprises -D₁-D₂-T₂ or -D₂-T₂,

D₁ is optionally present and if present, comprises an alkyl, a carbocyclic ring having 4 to 6 ring members or a heterocyclic ring having 4 to 6 ring members and 1,3 di-heteroatoms each heteroatom independently selected from O, S and N.

D₂ comprises an alkyl group having from one to about sixteen carbon atoms, CH═CH, C≡C, alkylamino, di-alkylamino, NH, a bicyclic ring, a tricyclic ring, 1-adamantyl -T₃, 2-adamantyl-T₃, adamantan-1-ylmethyl-T₃ or adamantan-2-ylidenemethyl -T₃.

T₂ comprises, in any possible position, a substituent group as later defined or —CO-T₄,
T₃ comprises an alkyl group having from 0 to about 9 carbon atoms,
T₄ comprises alkyl, C(halogen)₃ aminoalkyl, di-aminoalkyl, NH₂, a heterocyclic ring or a heteroaromatic ring.

Another embodiment of the disclosure comprises compound formula III, and physiologically acceptable salts thereof, 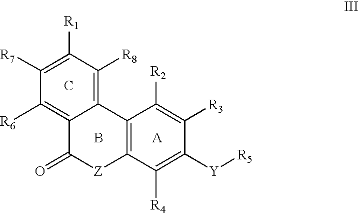
wherein:

Y comprises CH₂, CH(CH₃), C(CH₃)₂, a carbocyclic ring having 4 to 6 ring members or a heterocyclic ring having 4 to 6 ring members with 1 or 2 heteroatoms.

Z comprises O, S, NH, N-alkyl where alkyl comprises 1 to about 5 carbon atoms.

R₁ comprises H, halogen, N₃, NCS, CN, NO₂, NQ₁Q₂, ═O, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, ═CH₂, alkyl, alcohol, alkoxy, alkylmercapto, alkylamino, di-alkylamino or alkyl substituted in any possible position with at least one member selected from the substituent groups defined later.

R₂ comprises H, OH, OCH₃, OPO₃H₂, OSO₃H, PO₃H₂, SO₃H, halogen, C-(halogen)₃, NQ₁Q₂, COOQ₃, OQ3, NH—COalkyl, NH—COaryl, O—COalkyl, O—COalkyl-T₁, O—CO-T₁, alkyl-hydroxyl, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂
T₁ is in any possible position and comprises PO₃H, SO₃H, an alkyl group containing from 1 to about 16 carbons, tetrahydropyrrole, morpholine, thiomorpholine, piperazine, a heterocyclic ring or NQ₁Q₂;
   T₁ may be substituted in any possible position with at least one member selected from a substituent group, OPO₃H₂, OSO₃H, PO₃H₂, a heterocyclic ring or a heteroaromatic ring;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
Q₃ comprises H, alkyl, alcohol or alkyl-NQ₁Q₂.

R₃, R₄, R₆, R₇, or R₈ each independently comprise H, OH, halogen, C(halogen)₃, CN, N₃, NCS, NQ₁Q₂ or an alkyl group having 1 to about 4 carbon atoms,
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₅ comprises -D₁-D₂-T₂ or -D₂-T₂, with the below listed provisos.
D₁ is optionally present and if present, comprises an alkyl, a carbocyclic ring, a heterocyclic ring.
D₂ comprises an alkyl group having from one to about sixteen carbon atoms, CH═CH, C≡C, alkylamino, di-alkylamino, NH, a bicyclic ring, a tricyclic ring, 1-adamantyl-T₃, 2-adamantyl-T₃, adamantan-1-ylmethyl-T₃, or adamantan-2-ylidenemethyl-T₃,
T₂ comprises, in any possible position, a substituent group as later defined, —CO-T₄, a heterocyclic ring, a heterobicyclic ring structure, a heterotricyclic ring structure, a heteropolycyclic ring structure or a heteroaromatic ring with or without a substituent group as later defined.
T₃ comprises an alkyl group having from 0 to about 9 carbon atoms,
T₄ comprises H, C(halogen)₃, OH, NH₂, NO₂, alkyl, alkoxy, a heterocyclic ring or a heteroaromatic ring.

Provisos with respect to Structure Formula III:
When R₃, R₄, R₆, R₇ and R₈ are each H; R₁ is methyl; and R₂ is OH, then Y—R₅ can not be C(CH₃)₂(CH2)₅CH₃, CH(CH₂CH₃)₂ or CH2(CH₂)₃CH₃.
When R₃, R₄, R₆, R₇ and R₈ are each H; R₁ is methyl; and Y—R₅ is n-pentyl, then R₂ can not be OCOCH₃, OCH(CH₃)COCH₃, OCH₂CH(OC₂H₅)₂ or OCH₂CHO.
When R₃, R₄, R₆, R₇ and R₈ are each H; R₁ is bromide; and R₂ is OH, then Y—R₅ can not be n-pentyl.
When R₁ is CH₃; R₂ is OH; and one of R₇ and R₈ is OH and the other is H, Y—R₅ can not be n-pentyl.
When R₃, R₄, R₆, R₇ and R₈ are each H; formula III excludes compounds constructed by the combination of selecting R₁ from any of OH; OCH₃, OC₂H₅, OC₃H₇, OC₄H₉, and selecting Y—R₅ from any of (CH₂)_{q}CH ₃, C(CH₃)₂(CH₂)_{q}CH₃; (CH₂)_{q}—C≡C; C≡C(CH₂)_{q}; alkyl substituted adamantyl, as well as selecting Y from any five member ring and R₅ from (CH₂)_{q}CH₃, wherein q is an integer from 3-6.

In one advantageous variation, R₁ comprises halogen, C(halogen)₃, CH₂OH, a substituent group as later defined, an alkyl group having 1 to about 5 carbon atoms or an alkyl group having 1 to about 5 carbon atoms and substituted in any possible position with at least one member selected from substituent groups defined later.

R₂ comprises H, OH, OCH₃, OPO₃H₂, OSO₃H, PO₃H₂, SO₃H, halogen, C(halogen)₃, NQ₁Q₂, alkyl-hydroxyl, COOQ₃, OQ₃, NH—COalkyl, NH—COaryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂.
T₁ is in any possible position and comprises PO₃H, SO₃H, an alkyl group containing from 1 to about 16 carbons, tetrahydropyrrole, morpholine, thiomorpholine, piperazine, a heterocyclic ring or NQ₁Q₂;
   T₁ may be substituted in any possible position with at least one member selected from a substituent group, OPO₃H₂, OSO₃H, PO₃H₂, a heterocyclic ring or a heteroaromatic ring;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
Q₃ comprises H, alkyl, alcohol, or alkyl-NQ₁Q₂.

R₃, R₄, R₆, R₇ and R₈ comprises H, OH, halogen, C(halogen)₃, CN, N₃, NCS, NQ₁Q₂ or an alkyl group having 1 to about 4 carbon atoms,
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₅ comprises -D₁-D₂-T₂ or -D₂-T₂,

D₁ is optionally present and if present, comprises an alkyl group, a carbocyclic ring or a heterocyclic ring,
D₂ comprises an alkyl group having from one to about sixteen carbon atoms, CH═CH, C≡C, alkylamino, di-alkylamino, NH, a bicyclic ring, a tricyclic ring, 1-adamantyl-T₃, 2-adamantyl-T₃, adamantan-1-ylmethyl-T₃ or adamantan-2-ylidenemethyl-T₃,
T₂ comprises, in any possible position, a substituent group as later defined or —CO-T₄,
T₃ comprises an alkyl group having from 0 to about 9 carbon atoms,
T₄ comprises H, C(halogen)₃, OH, NH₂, alkylamino, di-alkylamino, NO₂, alkyl, alkoxy, a heterocyclic ring or a heteroaromatic ring.

Another embodiment of the disclosure comprises compound formula IV, and physiologically acceptable salts thereof, 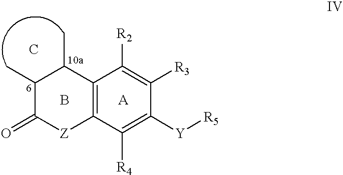
wherein:

The “C” Ring of compound formula IV comprises a carbocyclic ring, a bicyclic ring structure, a tricyclic ring structure, a heterocyclic ring, a heterobicyclic ring structure, or a heteroaromatic ring.

Y comprises CH₂, CHCH₃, C(CH₃)₂, a carbocyclic ring, an aromatic ring, a heterocyclic ring or a heteroaromatic ring,

Z comprises O, S, NH or N-alkyl.

R₂ comprises H, OH, OCH₃, OPO₃H₂, OSO₃H, P0₃H₂, SO₃H, halogen, C-(halogen) ₃, alkyl-hydroxyl, NQ₁Q₂, COOQ₃, OQ3, NH—COalkyl, NH—COaryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂.
T₁ is in any possible position and comprises PO₃H, SO₃H, an alkyl group containing from 1 to about 16 carbons, tetrahydropyrrole, morpholine, thiomorpholine, piperazine, a heterocyclic ring or NQ₁Q₂;
   T₁ may be substituted in any possible position with at least one member selected from a substituent group, OPO₃H₂, OSO₃H, P0₃H₂, a heterocyclic ring or a heteroaromatic ring;
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.
Q₃ comprises H, alkyl, alcohol, or alkyl-NQ₁Q₂.

R₃ and R₄ each independently comprise H, OH, halogen, C-(halogen)₃, CN, N₃, NCS, NQ₁Q₂ or an alkyl group having 1 to about 4 carbon atoms,
Q₁ and Q₂ each independently comprise H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members.

R₅ comprises -D₁-D₂-T₂ or -D₂-T₂, with the below listed provisos.
D₁ is optionally present and if present, comprises an alkyl, a carbocyclic ring or a heterocyclic ring.
D₂ comprises an alkyl group having from one to about sixteen carbon atoms, CH═CH, C≡C, alkylamino, di-alkylamino, NH, a bicyclic ring, a tricyclic ring, 1-adamantyl-T₃, 2-adamantyl-T₃, adamantan-1-ylmethyl-T₃, or adamantan-2-ylidenemethyl-T₃.
T₂ comprises, in any possible position, a substituent group as later defined, —CO-T₄, a heterocyclic ring, a heterobicyclic ring structure, a heterotricyclic ring structure, a heteropolycyclic ring structure or a heteroaromatic ring with or without a substituent group as later defined.
T₃ comprises an alkyl group having from 0 to about 9 carbon atoms,
T₄ comprises H, halogen, OH, NH₂, NO₂, alkyl, alkoxy, a heterocyclic ring or a heteroaromatic ring, with the proviso that when the C ring is 4-methyl cyclohexane with a double bond between the 6 and 10a positions, then Y—R₅ can not be a saturated alkyl group.

Provisos with respect to Structure Formula IV:

When the C ring is a pyridine or N-methyl-pyridine structure having the nitrogen in the para position to the carbonyl of B ring; R₃ and R₄ are hydrogen; then Y—R₅ can not be a straight or branched alkyl chain of 1 to 20 carbon atoms.

When the C ring is 4-methyl hexane having the methyl in the para position to the carbonyl of B ring; R₃ and R₄ are hydrogen; then Y—R₅ can not be CH₂COOH or a straight or branched chain alkyl of 1 to 20 carbon atoms.

When the C ring is a N-methyl tetrahydropyridine having a nitrogen in the para position to the carbonyl of the B ring; R₃ and R₄ are hydrogen; R₂ is OH; then Y—R₅ can not be OH, N-C₅H₁₁, CH(CH₃)(CH₂)₄CH₃, (CH2)11CH₃, or CH(cyclohexanyl).

When the C ring is a tetrahydropyridine having a nitrogen in the para position to the carbonyl of the B ring; R₃ and R₄ are hydrogen; Y—R₅ is 1.2-dimethylhexanyl; R₂ is OH; then the nitrogen of C ring can not be substituted with H, CHC₆H₆, CH₃ or CH₂C≡CH.

When the C ring is a N-benzyl-tetrahydropyridine having a nitrogen in the para position to the carbonyl of the B ring; R₃ and R₄ are hydrogen; R₂ is OH; then Y—R⁵ can not be CH(CH₃)CH₂COOCH₃, CH(CH₃)CH₂COOH, CH(CH₃)CH₂COCH₃, CH(CH₃)CH₂COOH CH₂CH₃ or CH(CH₃)CH₂C(CH₃)₂OH.

Unless otherwise specifically defined, “acyl” refers to the general formula —C(O)alkyl.

Unless otherwise specifically defined, “acyloxy” refers to the general formula —O-acyl.

Unless otherwise specifically defined, “alcohol” refers to the general formula alkyl-OH and includes primary, secondary and tertiary variations.

Unless otherwise specifically defined, “alkyl” or “lower alkyl” refers to a linear, branched or cyclic alkyl group having from 1 to about 16 carbon atoms including, for example, methyl, ethyl, propyl, butyl, hexyl, octyl, isopropyl, isobutyl, tert-butyl, cyclopropyl, cyclohexyl, cyclooctyl, vinyl and allyl. The alkyl group can be saturated or unsaturated. Unless otherwise specifically limited, an alkyl group can be unsubstituted, singly substituted or, if possible, multiply substituted, with substituent groups in any possible position. Unless otherwise specifically limited, a cyclic alkyl group includes monocyclic, bicyclic, tricyclic and polycyclic rings, for example norbornyl, adamantyl and related terpenes.

Unless otherwise specifically defined, “alkoxy” refers to the general formula —O-alkyl.

Unless otherwise specifically defined, “alkylmercapto” refers to the general formula —S-alkyl.

Unless otherwise specifically defined, “alkylamino” refers to the general formula —(NH)-alkyl.

Unless otherwise specifically defined, “di-alkylamino” refers to the general formula —N(alkyl)₂. Unless otherwise specifically limited di-alkylamino includes cyclic amine compounds such as piperidine and morpholine.

Unless otherwise specifically defined, an aromatic ring is an unsaturated ring structure having about 5 to about 7 ring members and including only carbon as ring atoms. The aromatic ring structure can be unsubstituted, singly substituted or, if possible, multiply substituted, with substituent groups in any possible position.

Unless otherwise specifically defined, “aryl” refers to an aromatic ring system that includes only carbon as ring atoms, for example phenyl, biphenyl or naphthyl. The aryl group can be unsubstituted, singly substituted or, if possible, multiply substituted, with substituent groups in any possible position.

Unless otherwise specifically defined, “aroyl” refers to the general formula —C(═O)-aryl.

Unless otherwise specifically defined, a bicyclic ring structure comprises 2 fused or bridged rings that include only carbon as ring atoms. The bicyclic ring structure can be saturated or unsaturated. The bicyclic ring structure can be unsubstituted, singly substituted or, if possible, multiply substituted, with substituent groups in any possible position. The individual rings may or may not be of the same type. Examples of bicyclic ring structures include, Dimethyl-bicyclo[3,1,1] heptane, bicyclo[2,2,1]heptadiene, decahydro-naphthalene and bicyclooctane.

Unless otherwise specifically defined, a carbocyclic ring is a non-aromatic ring structure, saturated or unsaturated, having about 3 to about 8 ring members that includes only carbon as ring atoms, for example, cyclohexadiene or cyclohexane. The carbocyclic ring can be unsubstituted, singly substituted or, if possible, multiply substituted, with substituent groups in any possible position.

Unless otherwise specifically defined, “fluorescence” refers to the emission of, or the property of emitting, electromagnetic radiation by a molecule resulting from and occurring only when that molecule is excited by the absorption of radiation from some other source.

Unless otherwise specifically defined, “halogen” refers to an atom selected from fluorine, chlorine, bromine and iodine.

Unless otherwise specifically defined, a heteroaromatic ring is an unsaturated ring structure having about 5 to about 8 ring members that has carbon atoms and one or more heteroatoms, including oxygen, nitrogen and/or sulfur, as ring atoms, for example, pyridine, furan, quinoline, and their derivatives. The heteroaromatic ring can be unsubstituted, singly substituted or, if possible, multiply substituted, with substituent groups in any possible position.

Unless otherwise specifically defined, a heterobicyclic ring structure comprises 2 fused or bridged rings that include carbon and one or more heteroatoms, including oxygen, nitrogen and/or sulfur, as ring atoms. The heterobicyclic ring structure is saturated or unsaturated. The heterobicyclic ring can be unsubstituted, singly substituted or, if possible, multiply substituted, with substituent groups in any possible position. The individual rings may or may not be of the same type. Examples of heterobicyclic ring structures include tropane, quinuclidine and tetrahydro-benzofuran.

Unless otherwise specifically defined, a heterocyclic ring is a saturated or unsaturated ring structure having about 3 to about 8 ring members that has carbon atoms and one or more heteroatoms, including oxygen, nitrogen and/or sulfur, as ring atoms, for example, piperidine, morpholine, piperazine, pyrrolidine, thiomorpholine, tetrahydropyridine, and their derivatives. The heterocyclic ring can be unsubstituted, singly substituted or, if possible, multiply substituted, with substituent groups in any possible position.

Unless otherwise specifically defined, a heterotricyclic ring structure comprises 3 rings that may be fused, bridged or both fused and bridged, and that include carbon and one or more heteroatoms, including oxygen, nitrogen and/or sulfur, as ring atoms. The heterotricyclic ring structure can be saturated or unsaturated. The heterotricyclic ring structure can be unsubstituted, singly substituted or, if possible, multiply substituted, with substituent groups in any possible position. The individual rings may or may not be of the same type. Examples of heterotricyclic ring structures include 2,4,10-trioxaadamantane, tetradecahydro-phenanthroline.

Unless otherwise specifically defined, a heteropolycyclic ring structure comprises more than 3 rings that may be fused, bridged or both fused and that include carbon and one or more heteroatoms, including oxygen, nitrogen and/or sulfur, as ring atoms. The heteropolycyclic ring structure can be saturated or unsaturated. The heteropolycyclic ring structure can be unsubstituted, singly substituted or, if possible, multiply substituted, with substituent groups in any possible position. The individual rings may or may not be of the same type. Examples of heteropolycyclic ring structures include azaadamantine, 5-norbornene-2,3-dicarboximide.

Unless otherwise specifically defined, the term “phenacyl” refers to the general formula -phenyl-acyl.

Unless otherwise specifically defined, a polycyclic ring structure comprises more than 3 rings that may be fused, bridged or both fused and bridged, and that includes carbon as ring atoms. The polycyclic ring structure can be saturated or unsaturated. The polycyclic ring structure can be unsubstituted, singly substituted or, if possible, multiply substituted, with substituent groups in any possible position. The individual rings may or may not be of the same type. Examples of polycyclic ring structures include adamantine, bicyclooctane, norbornane and bicyclononanes.

Unless otherwise specifically defined, a spirocycle refers to a ring system wherein a single atom is the only common member of two rings. A spirocycle can comprise a saturated carbocyclic ring comprising about 3 to about 8 ring members, a heterocyclic ring comprising about 3 to about 8 ring atoms wherein up to about 3 ring atoms may be N, S, or O or a combination thereof.

Unless otherwise specifically defined, a tricyclic ring structure comprises 3 rings that may be fused, bridged or both fused and bridged, and that includes carbon as ring atoms. The tricyclic ring structure can be saturated or unsaturated. The tricyclic ring structure can be unsubstituted, singly substituted or, if possible, multiply substituted, with substituent groups in any possible position, and may be substituted or unsubstituted. The individual rings may or may not be of the same type. Examples of tricyclic ring structures include fluorene and anthracene.

Unless otherwise specifically limited the term substituted means substituted by a below described substituent group in any possible position. Substituent groups for the above moieties useful in the disclosure are those groups that do not significantly diminish the biological activity of the disclosed compound. Unless otherwise specifically limited a substituent group or a substituent group that does not significantly diminish the biological activity of the disclosed compound includes, for example, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, COOX₃, SO₃H, PO₃H₂, SO₂NX₁X₂, CONX₁X₂, COC(halogen)₃, alkyl, alcohol, alkoxy, alkylmercapto, alkylamino, di-alkylamino, sulfonamide or thioalkoxy wherein X₁ and X₂ each independently comprise H or alkyl, or X₁ and X₂ together comprise part of a heterocyclic ring having about 4 to about 7 ring members and optionally one additional heteroatom selected from O, N or S, or X₁ and X2 together comprise part of an imide ring having about 5 to about 6 members and X₃ comprises H, alkyl, loweralkylhydroxy, or alkyl-NX₁X₂. Unless otherwise specifically limited, a substituent group may be in any possible position.

An isotope is one of two or more species of the same element. Each isotope of an element will have the same number of protons in its nucleus, the same atomic number and the same position in the Periodic Table. However each isotope of that element will have a different number of neutrons in its nucleus and therefore a different mass than other isotopes of that species. The term nuclide is sometimes used synonymously with the term isotope. As used herein a natural isotope has an atomic mass corresponding most closely with the atomic mass shown for that element in the Periodic Table. As used herein an unnatural isotope has an atomic mass that is further removed from the atomic mass shown for that element in the Periodic Table than the natural isotope. For example, protium (hydrogen-1 or ¹H) is the natural isotope of hydrogen and deuterium (hydrogen-2 or ²H) and tritium (hydrogen-3 or ³H) are all unnatural isotopes of hydrogen. The compounds of the present disclosure can comprise isotopes at one or more of their atoms. For example, the compounds can be radiolabeled with isotopes, such as tritium, carbon-11, carbon-13, carbon-14, oxygen-15, nitrogen-15, oxygen-18, fluorine-18, bromine-76, bromine-77, bromine-82, iodine-123 or iodine-125. The present disclosure encompasses all isotopic variations of the described compounds, whether natural or unnatural, radioactive or not.

In general, unless otherwise explicitly stated the disclosed materials may be alternately formulated to comprise, consist of, or consist essentially of, any appropriate components or moieties herein disclosed. The disclosed materials may additionally, or alternatively, be formulated so as to be devoid, or substantially free, of any components, materials, ingredients, adjuvants moieties or species used in the prior art compositions or that are otherwise not necessary to the achievement of the function and/or objective of the present disclosure.

When the word “about” is used herein it is meant that the amount or condition it modifies can vary some beyond the stated amount so long as the function and/or objective of the disclosure are realized. The skilled artisan understands that there is seldom time to fully explore the extent of any area and expects that the disclosed result might extend, at least somewhat, beyond one or more of the disclosed limits. Later, having the benefit of this disclosure and understanding the concept and embodiments disclosed herein, a person of ordinary skill can, without undue effort, explore beyond the disclosed limits and, when embodiments are found to be without any unexpected characteristics, those embodiments are within the meaning of the term about as used herein.

Some of the disclosed cannabinoid compounds exhibit high affinity for the CB1 and/or CB2 cannabinoid receptors. Thus, another aspect of the disclosure is use of at least one of the disclosed compounds to interact with cannabinoid receptors.

Further, some of the disclosed cannabinoid compounds show a surprisingly higher selectivity for one of the cannabinoid receptors. These selective compounds are able to interact with one cannabinoid receptor, for example the CB2 cannabinoid receptor, without affecting the other cannabinoid receptor to the same degree. Therefore, still another aspect of the disclosure is use of at least one of the disclosed compounds to preferentially interact with one cannabinoid receptor.

Some of the disclosed compounds exhibit fluorescence properties. Therefore, still another aspect of the disclosure is the use of the fluorescent properties of cannabinoid compounds. In a variation, the fluorescence properties allow qualitative or quantitative detection of the cannabinoid compounds and or their complex with cannabinoid receptors .

Some of the disclosed fluorescent cannabinoid compounds exhibit high affinity and/or selectivity for the CB1 and/or CB2 cannabinoid receptors. Therefore, still another aspect of the disclosure is a method of using the fluorescent cannabinoid compounds as fluorescent biosensors. In some embodiments the disclosed method is capable of sensing and reporting the bio-behaviors of cannabinoid receptors and molecules associated with the cannabinoid receptors through a variety of fluorescence technologies. Some applicable fluorescence technologies useful with the disclosed method include, for example, Fluorescence Microscopy, Fluorescence Polarization Spectroscopy, Fluorescence Resonance Energy Transfer Analysis, Flow Cytometry, Fluorescence Photo-Bleach, Immunofluorescence, and Fluorescent Competitive Binding Assay. It should be understood that the present method encompasses use of the disclosed compounds in any technology wherein their fluorescent properties are desirable. Thus, the disclosed fluorescent cannabinoids can be employed as Fluorescent Molecular Probes, Fluorescent Imaging Agents, Fluorescent Control Standards and Cellular Markers in a broad scope of biomedical research involving cannabinoid receptors. In addition, the fluorescent cannabinoids can be applied in clinical use as Fluorescent Diagnostic Agents to determine therapeutic drug levels and the presence of drugs of abuse in fluids. The fluorescent cannabinoids can also be used as diagnostic agents for determination of white blood cells that have a high concentration of CB2 receptors.

Some of the disclosed cannabinoid compounds can act as high affinity modulators for cannabinoid receptors. The disclosed cannabinoid compounds therefore are potential therapeutic agents through the modulation of the CB1 and/or CB2 cannabinoid receptors.

Some of the novel cannabinoid compounds described herein may be cannabinoid receptor agonists. The disclosed cannabinoid agonists interact with the CB1 and/or CB2 cannabinoid receptor binding site to initiate a physiological or a pharmacological response characteristic of that receptor. Therefore, a further aspect of the disclosure is use of at least one of the compounds to initiate an agonistic response from a cannabinoid receptor.

Some of the novel cannabinoid compounds described herein may be cannabinoid receptor antagonists. The disclosed cannabinoid antagonists interact with the CB1 and/or CB2 cannabinoid receptor binding site to block other ligands from the receptor binding site without initiating a physiological or a pharmacological response characteristic of that receptor. Thus, cannabinoid antagonists typically oppose the cannabinoid receptor site response characteristics initiated by cannabinoid agonists. Therefore, a further aspect of the disclosure is use of at least one of the compounds to oppose initiation of an agonistic response from a cannabinoid receptor.

The disclosed cannabinoid compounds described herein, and physiologically acceptable salts thereof, have pharmacological properties when administered in therapeutically effective amounts for providing a physiological response in individuals and/or animals. Thus, another aspect of the disclosure is the administration of a therapeutically effective amount of at least one of the compounds, or a physiologically acceptable salt thereof, to an individual or animal to provide a physiological response.

A better understanding of the disclosure will be obtained from the following detailed description of the article and the desired features, properties, characteristics, and the relation of the elements as well as the process steps, one with respect to each of the others, as set forth and exemplified in the description and illustrative embodiments.

### DETAILED DESCRIPTION

The use of radiolabeled cannabinoid compounds in research (for example in binding assays) is well known, as are the problems with obtaining, using and disposal of radioactive compounds. The disclosed fluorescent cannabinoid compounds can be used to conduct research in a similar manner as radiolabeled compounds. However, fluorescent techniques such as, for example, Fluorescence Resonance Energy Transfer (FRET) can be used to assess results of the research in place of radiocounting techniques. Naturally, the disclosed compounds provide benefits in procurement, handling and disposal over radioactive compounds. Additionally, fluorescence techniques are highly specific and sensitive, so that the disclosed compounds can provide improvements in specificity and sensitivity over radiotechniques.

Fluorescent ligands are generally useful to probe and sense receptor mechanism include the histochemical localization of receptors, their visualization on cell surface, quantification of receptor mobility by the technique of fluorescence recovery after photobleaching, and fluorescent energy transfer experiments to characterize the receptor environment, e.g. the lipid bilayer in membrane or the topolography of binding sites on isolated receptor molecules.

The potential applications of the disclosed fluorescent cannabinoids as biosensor, molecular probe, cellular marker and imaging agent can be extrapolated from numerous published works employing fluorescent ligands in other, non-cannabinoid biological system. For instance, Ariano M. A. et al described the visualization of dopamine receptors by using fluorescent ligands as imaging agents (Multiple fluorescent ligands for dopamine receptors. II. Visualization in neural tissues. Brain Res. 1991 May 3;547(2):208-22), Melamed E. reported the visualization of beta-adrenoceptor in rat cerebellum via a fluorescent analogue of propranolol (Direct localisation of beta-adrenoceptor sites in rat cerebellum by a new fluorescent analogue of propranolol, Nature. 1976 Jun. 3;261(5559):420-2), and Miksicek R J et al described the imaging of estrogen receptors by a fluorescent ligand (In situ localization of the estrogen receptor in living cells with the fluorescent phytoestrogen coumestrol. J Histochem Cytochem. 1993 June;41(6):801-10). Furthermore, applications of fluorescent compounds as molecular probes or biosensors can be exemplified by the work of McCabe R T (Characterization of benzodiazepine receptors with fluorescent ligands, FASEB J. 1990 August;4(11):2934-40); Vallotton P, (Mapping The Antagonist Binding Site of The Serotonin Type 3 Receptor by Fluorescence Resonance Energy Transfer, Biochemistry, 2001 Oct. 16;40(41):12237-42); Jones G, (Azole-linked coumarin dyes as fluorescence probes of domain-forming polymers, J Photochem Photobiol B. 2001 Dec. 1;65(1):5-12); Vallotton P et al (In Vitro and In Vivo Ligand Binding to the 5HT(3) Serotonin Receptor Characterised by Time-Resolved Fluorescence Spectroscopy, Chembiochem Europ J Chem Biol. 2001 Mar. 2;2(3):205-11), Epand RF, (Fluorescent probes of membrane surface properties, Biochim Biophys Acta, 1996 Oct. 23; 1284(2):191-5); Balice-Gordon R J (In vivo observations of pre- and postsynaptic changes during the transition from multiple to single innervation at developing neuromuscular junctions, J Neurosci. 1993 February;13(2):834-55) Yamamoto T (Spectroscopic monitoring of local conformational changes during the intramolecular domain-domain interaction of the ryanodine receptor, Biochemistry. 2002 Feb. 5;41(5):1492-501); Janssen M J et al (A fluorescent receptor assay for benzodiazepines using coumarin-labeled desethylflumazenil as ligand, Anal Chem. 2001 Jul. 1;73(13):3168-73), Guatimosim C etal (Use of fluorescent probes to follow membrane traffic in nerve terminals. Braz J Med Biol Res. 1998 November;31(11):1491-500), and Hazum E (Cluster formation of opiate (enkephalin) receptors in neuroblastoma cells: differences between agonists and antagonists and possible relationships to biological functions, Proc Natl Acad Sci U S A. 1980 May;77(5):3038-41).

Some fluorescent compounds have also been used as diagnostic agents. For example, Cortvrindt RG et al described a method of using a non-cannabinoid fluorescent compound to detect follicle density and staging in human ovarian cortical biopsy samples (Fluorescent probes allow rapid and precise recording of follicle density and staging in human ovarian cortical biopsy samples, Fertil Steril. 2001 March;75(3):588-93), Collins A K et al suggested a method of employing a coumarin fluorescent product as radiation dosimeter in radiation therapy (Coumarin chemical dosimeter for radiation therapy, Med Phys. 1994 November;21(11):1741-7), and Nairn RC et al described the usefulness of fluorescent probes in monitoring cell immunity (Fluorescent probes for rapid tests of cellular immunoreactivity, Pathology. 1984 January;16(1):1-3).

The disclosed fluorescent cannabinoids can also be used as a diagnostic tool to label and measure cells containing cannabinoid receptors. In human leukocytes, the CB2 receptors are found with particularly high abundance on B-cells, natural killer cells and macrophages. Leukocytes are the cells responsible for immunosurveillance and for the specificity of immune defense in humans. The quantification of the major types of human leukocytes has proved to be of great diagnostic and prognostic value in different pathologic conditions. As one example, count of peripheral blood natural killer cells is suggested as a useful index in prognosis of large cell lymphoma (Baumann MA et al, Correlation of circulating natural killer cell count with prognosis in large cell lymphoma, Cancer. 1986 Jun. 15;57(12):2309-12). Typically, quantification of lymphocytes employs immuno-fluorescent antibodies as lymphocyte markers. Therefore, a potential usefulness of this disclosure is that at least one disclosed compound can be added to white blood cells to bind to the CB2 receptors therein. Fluorescent measurement techniques can be used to qualitatively and/or quantitatively assess the compounds present and thereby label the cannabinoid receptors and provide information as to the quantity of receptors and white blood cells. The disclosed fluorescent cannabinoids can also be used as an imaging agent. Addition of at least one disclosed compound to a tissue sample allows binding of the compound to receptors therein. Subsequent excitation of the bound compound/tissue sample allows image analysis of the emitted light.

As used herein a “therapeutically effective amount” of a compound, is the quantity of a compound which, when administered to an individual or animal, results in a sufficiently high level of that compound in the individual or animal to cause a physiological response. The compounds described herein, and physiologically acceptable salts thereof, have pharmacological properties when administered in therapeutically effective amounts for providing a physiological response useful to treat central and peripheral pain, neuropathy, neurodegenerative diseases including multiple sclerosis, Parkinson's disease, Huntington's chorea, Alzheimer's disease; mental disorders such as schizophrenia and depression; to prevent or reduce endotoxic shock and hypotensive shock; to modulate appetite; to modulate the immune system; to reduce fertility; to prevent or reduce diseases associated with motor function such as Tourette's syndrome; to prevent or reduce inflammation; to provide neuroprotection; to suppress memory; to produce peripheral vasodilation; to treat epilepsy, glaucoma, nausea associated with cancer chemotherapy as well as other ailments in which cannabinoid system is implicated.

The compounds described herein, and physiologically acceptable salts thereof, have pharmacological properties when administered in therapeutically effective amounts for providing a physiological response useful to treat nausea associated with Aids wasting syndrome or to enhance appetite in AIDS wasting syndrome.

Typically, a “therapeutically effective amount” of a disclosed compound is believed to range from about 5 mg/day to about 1,000 mg/day.

As used herein, an “individual” refers to a human. An “animal” refers to, for example, veterinary animals, such as dogs, cats, horses and the like, and farm animals, such as cows, pigs and the like.

The compound of the present disclosure can be administered by a variety of known methods, including, for example, orally, rectally, or by parenteral routes (e.g., intramuscular, intravenous, subcutaneous, nasal or topical). The form in which the compounds are administered will be determined by the route of administration. Such forms include, but are not limited to, capsular and tablet formulations (for oral and rectal administration), liquid formulations (for oral, intravenous, intramuscular, subcutaneous, ocular, intranasal, inhalation-based and transdermal administration) and slow releasing microcarriers (for rectal, intramuscular or intravenous administration). The formulations can also comprise one or more of a physiologically acceptable excipient, vehicle and optional adjuvants, flavorings, colorants and preservatives. Suitable physiologically acceptable vehicles include, for example, saline, sterile water, Ringer's solution and isotonic sodium chloride solutions. The specific dosage level of active ingredient will depend upon a number of factors, including, for example, biological activity of the particular preparation, age, body weight, sex and general health of the individual being treated.

The following examples are given for purposes of illustration only in order that the present disclosure may be more fully understood. These examples are not intended to limit in any way the scope of the disclosure unless otherwise specifically indicated.

### EXAMPLES

TABLE 1 illustrates some synthesized cannabinoids (compounds 1-56). Compounds 1-27, 38 and 45-46 are representative fluorescent cannabinoids.

TABLE 1

| | |
|---|---|
| | 1 |
| 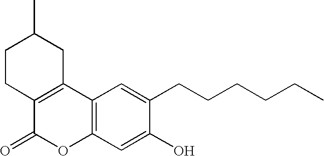 | |
| | 2 |
| 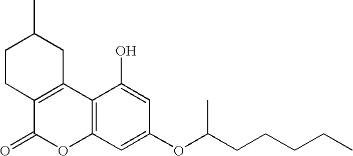 | |
| | 3 |
| 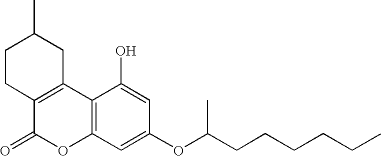 | |
| | 4 |
| 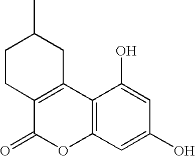 | |
| | 5 |
| 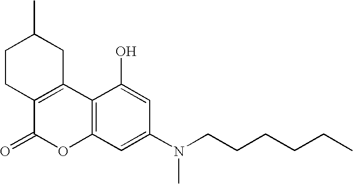 | |
| | 6 |
| 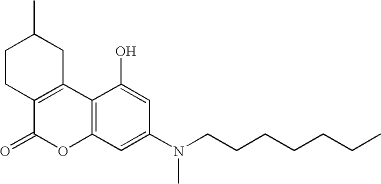 | |
| | 7 |
| 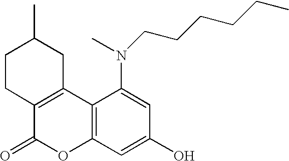 | |
| | 8 |
| 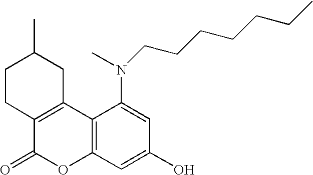 | |
| | 9 |
| 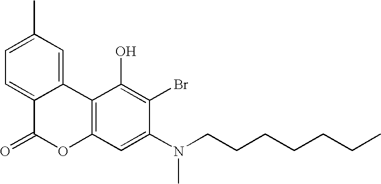 | |
| | 10 |
| 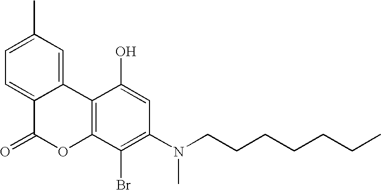 | |
| | 11 |
| 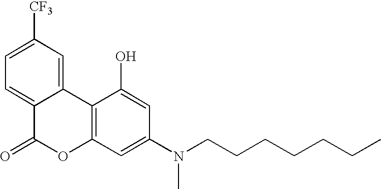 | |
| | 12 |
| 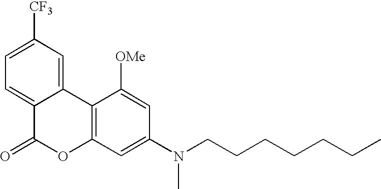 | |
| | 13 |
| 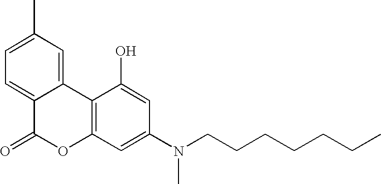 | |
| | 14 |
| 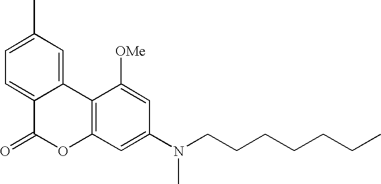 | |
| | 15 |
| 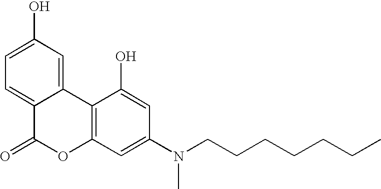 | |
| | 16 |
| 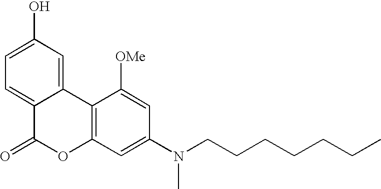 | |
| | 17 |
| 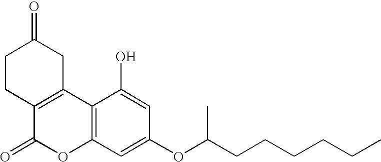 | |
| | 18 |
| 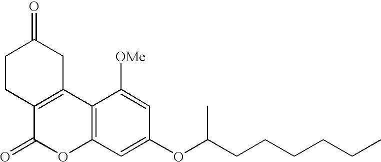 | |
| | 19 |
| 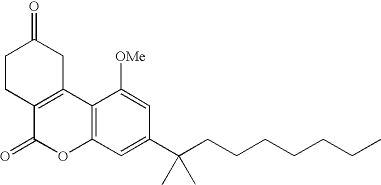 | |
| | 20 |
| 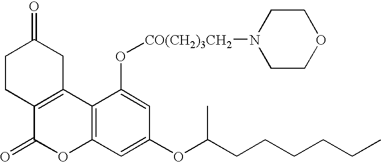 | |
| | 21 |
| 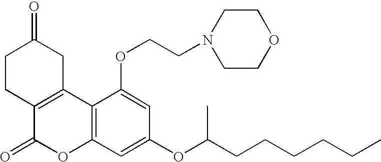 | |
| | 22 |
| 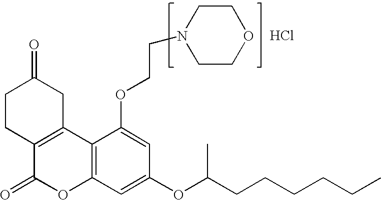 | |
| | 23 |
| 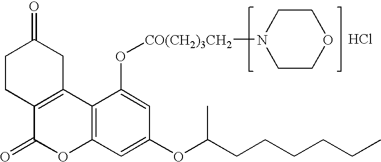 | |
| | 24 |
| 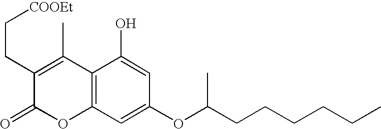 | |
| | 25 |
| 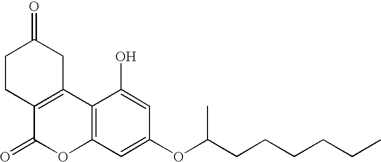 | |
| | 26 |
| 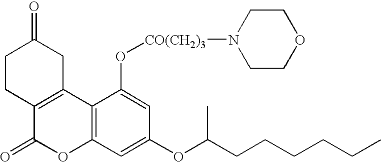 | |
| | 27 |
| 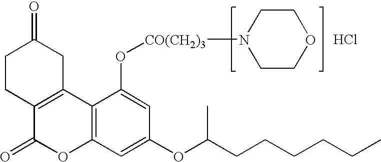 | |
| | 28 |
| 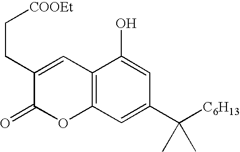 | |
| | 29 |
| 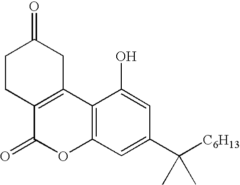 | |
| | 30 |
| 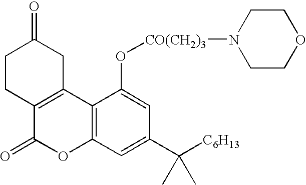 | |
| | 31 |
| 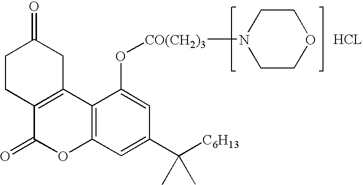 | |
| | 32 |
| 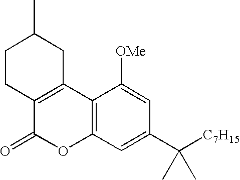 | |
| | 33 |
| 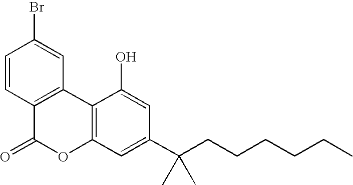 | |
| | 34 |
| 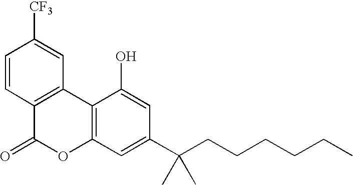 | |
| | 35 |
| 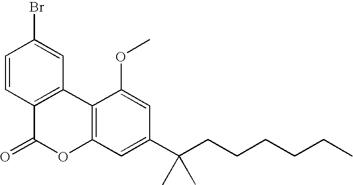 | |
| | 36 |
| 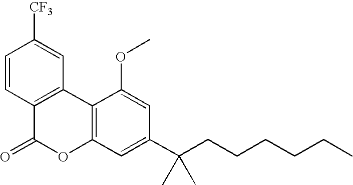 | |
| | 37 |
| 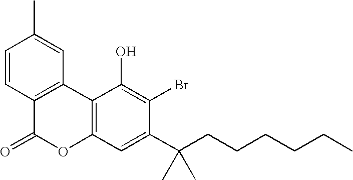 | |
| | 38 |
| 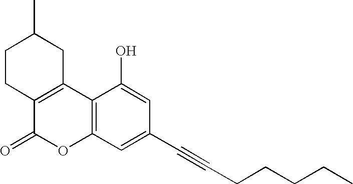 | |
| | 39 |
| 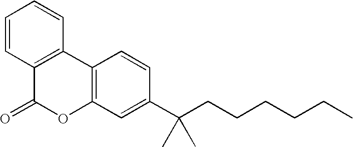 | |
| | 40 |
| 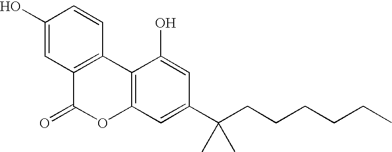 | |
| | 41 |
| 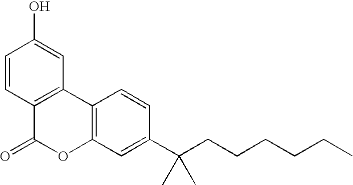 | |
| | 42 |
| 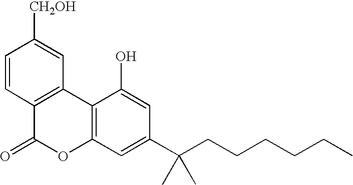 | |
| | 43 |
| 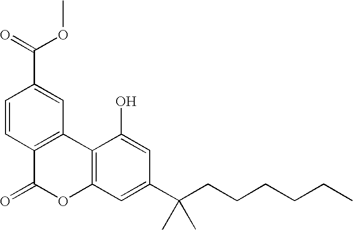 | |
| | 44 |
| 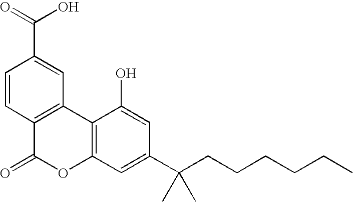 | |
| | 45 |
| 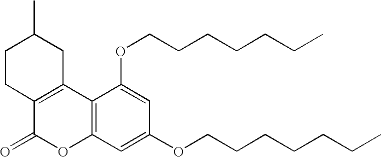 | |
| | 46 |
| 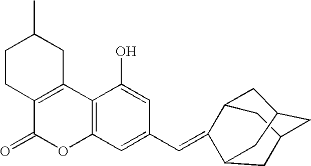 | |
| | 47 |
| 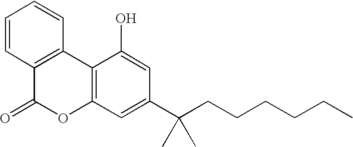 | |
| | 48 |
| 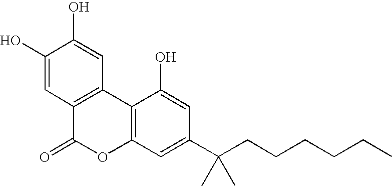 | |
| | 49 |
| 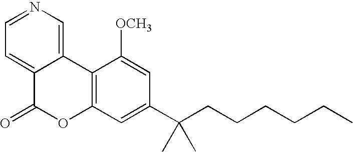 | |
| | 50 |
| 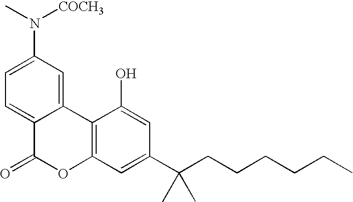 | |
| | 51 |
| 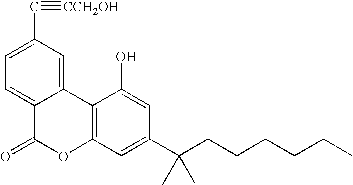 | |
| | 52 |
| 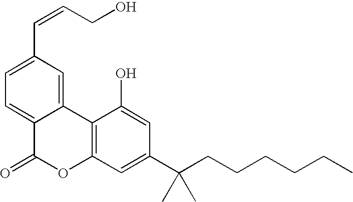 | |
| | 53 |
| 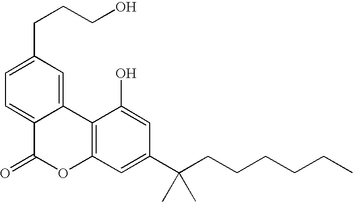 | |
| | 54 |
| 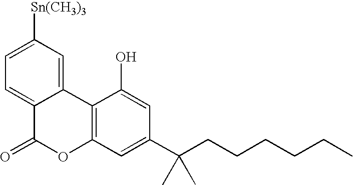 | |
| | 55 |
| 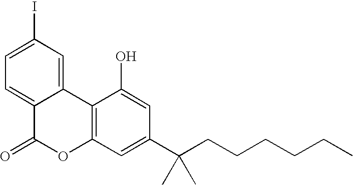 | |
| | 56 |
| 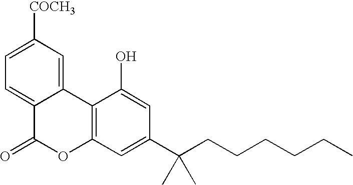 | |

The fluorescent properties of some of the disclosed compounds were examined using the following fluorescence assay protocol. All samples were processed in the same manner and diluted in J T BAKER PHOTREX Grade MeOH. Cuveftes and flasks were cleaned and rinsed with distilled water then rinsed twice with MeOH. Samples were kept in the freezer prior to testing. NSG Precision Quartz Cuveftes were used for absorption and spectral scans. Samples were diluted with methanol to a final volume of 5 mL. A 100× dilution of the samples in MeOH was performed. Absorption spectra were run using a Beckman DU-40 Spectrophotometer. Guided by absorption spectra, excitation and emission scans were run using an ISA Fluoromax-2 Fluorometer. 500 μL of MeOH was added into sample vial containing about 1 mg of testing compound, and aspirated with pipette to mix. 500 μL sample aliquot was then placed into 5 mL volumetric flask and brought up to volume with MeOH, and 5 mL was used as stock. Serial dilutions in 50 mL volumetric flasks were performed until final concentration of 100× reached. Dilution protocol was repeated for each sample. After dilution, 2.5 mL of sample was placed into a 3 mL Quartz cuvefte and the cuvette was placed in ISA Fluoromax-2 Fluorometer. Absorption scan was run in Beckman DU-40 Spectrophotometer. Excitation and emission scans were run and spectral data were collected. Some of the disclosed fluorescent cannabinoid compounds exhibit strong fluorescence in the ultraviolet and visible wavelength ranges. Representative fluorescent data for some disclosed compounds is shown in TABLE 2.

TABLE 2

| | | | Fluo- | Fluo- | |
|---|---|---|---|---|---|
| | Absor- | Mol | rescence | rescence | |
| | bance | Extinction | Excitation | Emission | Stoke's |
| com- | Peak | 1/((Mol × | Peak | Peak | Shift |
| pound | (nm) | cm)/L) | (nm) | (nm) | (nm) |
| 1 | 327 | 1.50E+04 | 330 | 390 | 60 |
| 2 | 320 | 1.39E+04 | 326 | 423 | 97 |
| 5 | 364 | 1.86E+04 | 368 | 460 | 92 |
| 6 | 364 | 1.80E+04 | 367 | 460 | 93 |
| 8 | 405 | 1.14E+04 | 405 | 465 | 60 |
| 13 | 316 | 2.27E+04 | 319 | 519 | 200 |
| 16 | 317 | 2.18E+04 | 321 | 498 | 177 |
| 15 | 318 | 2.34E+04 | 320 | 502 | 182 |

Some of the disclosed compounds were tested for CB2 receptor binding affinity and for CB1 receptor affinity (to determine selectivity for the CB2 receptor). As used herein, “binding affinity” is represented by the Kᵢ value which is the inhibition constant correlated with the concentration of an analog required to occupy the 50% of the total number (Bmax) of the receptors. The lower the Kᵢ value the higher the binding affinity. As used herein an analog is said to have “binding selectivity” if it has higher binding affinity for one receptor compared to the other receptor; e.g. a cannabinoid analog which has a Kᵢ of 0.1 nM for CB1 and 10 nM for CB2, is 100 time more selective for the CB1 receptor. For the CB1 receptor binding studies, membranes were prepared from rat forebrain membranes according to the procedure of P. R. Dodd et al, A Rapid Method for Preparing Synaptosomes: Comparison with Alternative Procedures, Brain Res., 107-118 (1981). The binding of the novel analogues to the CB1 cannabinoid receptor was assessed as described in W. A. Devane et al, Determination and Characterization of a Cannabinoid Receptor in a Rat Brain, Mol. Pharmacol., 34, 605-613 (1988) and A. Charalambous et al, 5′-azido Δ⁸⁻THC: A Novel Photoaffinity Label for the Cannabinoid Receptor, J. Med. Chem., 35, 3076-3079 (1992) with the following changes. The above articles are incorporated by reference herein.

Membranes, previously frozen at −80° C., were thawed on ice. To the stirred suspension was added three volumes of TME (25 mM Tris-HCl buffer, 5 mM MgCI₂ and 1 mM EDTA) at a pH 7.4. The suspension was incubated at 40° C. for 30 min. At the end of the incubation, the membranes were pelleted and washed three times with TME.

The treated membranes were subsequently used in the binding assay described below. Approximately 30 μg of membranes were incubated in silanized 96-well microtiter plate with TME containing 0.1% essentially fatty acid-free bovine serum albumin (BSA), 0.8 nM [³H] CP-55,940, and various concentrations of test materials in a final volume of 200 μL. The assays were incubated for 1 hour at 30° C. and then immediately filtered using Packard Filtermate 196 harvester and Whatman GF/C filterplates and washed with wash buffer (TME) containing 0.5% BSA. Radioactivity was detected using MicroScint 20 scintillation cocktail added directly to the dried filterplates, and the filterplates were counted using a Packard Instruments Top-Count. Nonspecific binding was assessed using 100 nM CP-55,940. Data collected from three independent experiments performed with duplicate determinations was normalized between 100% and 0% specific binding for [³H] CP-55,940, determined using buffer and 100 nM CP-55,940. The normalized data was analyzed using a 4-parameter nonlinear logistic equation to yield IC₅₀ values. Data from at least two independent experiments performed in duplicate was used to calculate IC₅₀ values which were converted to Kᵢ values using the assumptions of Cheng et al, Relationship Between the Inhibition Constant (Kᵢ) and the concentration of Inhibitor which causes 50% Inhibition (IC₅₀) of an Enzymatic Reaction, Biochem. Pharmacol., 22, 3099-3102, (1973), which is incorporated by reference herein.

For the CB2 receptor binding studies, membranes were prepared from frozen mouse spleen essentially according to the procedure of P. R. Dodd et al, A Rapid Method for Preparing Synaptosomes: Comparison with Alternative Procedures, Brain Res., 226, 107-118 (1981) which is incorporated by reference herein. Silanized centrifuge tubes were used throughout to minimize receptor loss due to adsorption. The CB2 binding assay was conducted in the same manner as for the CB1 binding assay. The binding affinities (Kᵢ) were also expressed in nanomoles (nM). Some of the disclosed compounds illustrated in TABLE 1 showed high affinities for The CB1 and CB2 cannabinoid receptors, with CB1 Kᵢ values as low as 6.0 nM and CB2 Kᵢ values as low as 0.6 nM. Some of the disclosed compounds show CB2/CB1 selectivity of 760 in favor of the CB2 receptor. Table 3 illustrates CB1 and CB2 Kᵢ values for some disclosed compounds.

TABLE 3

| Compound | CB1 (nM) | CB2 (nM) |
|---|---|---|
| 1 | 8610 | 5630 |
| 2 | 150 | 25 |
| 3 | 30 | 6 |
| 17 | 4185 | 1233 |
| 20 | 2003 | 1661 |
| 21 | 122 | 128 |
| 22 | 160 | 288 |
| 23 | 2577 | 824 |
| 5 | 50 | 3 |
| 6 | 9 | 0.7 |
| 7 | 6680 | 1685 |
| 8 | 6365 | 1479 |
| 13 | 140 | 16 |
| 9 | 1264 | 90 |
| 10 | 990 | 23 |
| 14 | 1305 | 148 |
| 15 | 304 | 0.4 |
| 16 | 8434 | 355 |
| 11 | 40 | 1 |
| 47 | 4787 | 6.6 |
| 48 | 1946 | 100.6 |
| 49 | 3758 | 35.4 |

Preparation of Compounds

General All the reagents and solvents used in the following reactions are available from Sigma-Aldrich Fine Chemicals of Milwaukee, Wis. and/or Lancaster Synthesis Inc. of Windham, N.H. at the highest available grade except where indicated. All reactions were carried out under scrupulously dry conditions unless otherwise stated. Work-up organic phases are dried over Na₂SO₄, and solvents are then removed under reduced pressure. Column chromatography was carried out by using active silica gel (230-400 mesh) available from Selecto Scientific of Suwanee, Ga. All compounds are demonstrated to be homogeneous by analytical TLC on pre-coated silica gel TLC plates (Whatman Ltd, Maidstone, Kent, England), and chromatograms are visualized by phosphomolybdic acid staining and UV lamp at wavelength of 254 nm. Structures are determined by ¹H NMR spectra recorded on Bruker DMX-500 MHz spectometers and Mass spectra obtained on a Hewlett Packard HP 6890 GC-MS instrument.

The disclosed compounds were prepared generally using three types of reactions, von Pechmann Condensation, Oxazoline facilitated aromatic carbon-carbon coupling, and Suzuki Coupling Reaction. The preparation procedures include aspects of the following references. Any reference cited in this disclosure is hereby incorporated by reference.

Alo, B. I.; Kandil, A.; Patil, P. A.; Sharp, M. J.; Siddiqui, M. A.; and Snieckus, V. Sequential Directed Ortho Metalation-Boronic Acid Cross-Coupling Reactions. A general Regiospecific Route to Oxygenerated Dibenzo[b,d]pyran-6-ones Related to Ellagic Acid, J. Org. Chem. 1991, 56, 3763-3768. Beak, P.; and Brown, R A., The Tertiary Amide as an Effective Director of Ortho Lithiation, J. Org. Chem. 1982, 47, 34-36. Watanabe, T.; Miyaura, N.; Suzuki, A., Synthesis of Sterically Hindered Biaryls via the Palladium Catalyzed Cross-Coupling Reaction of Arylboronic Acids or their Esters with Haloarenes, Synlett 1992, 207-210. Morris, S,; Mechoulam, R.; and Irene, Y., Halogenation of phenols and Phenyl ethers with Potassium Halides in the Presence of 18-Crown-6 on Oxidation with m-Chloroperbenzoic Acid, J. Chem. Soc., Perkin Trans. 1 1987, 1423-1427. Rhee, M. H.; Vogel, Z.; Barg, J.; Bayewitch, M.; Levy, R.; Hanus, L.; Breuer, A.; and Mechoulam, R., Cannabinol Derivatives: Binding to Cannabinoid Receptors and Inhibition of Adenylcyclase, J. Med. Chem. 1997, 40, 3228-3233. Fahrenholtz, K. E., Lurie, M. and Kierstead, A R. W., The Total Synthesis of dl-Δ⁹-Tetrahydrocannabinol and Four of Its Isomers, J. Amer. Chem. Soc. 1967, 89:23, 5934-5941. Love, R. Bender, P. E., Dowalo, F., Macko, E., and Fowler, P., Cannabinoids. Structure-Activity Studies Related to 1,2-Dimethylheptyl Derivatives, J. Med. Chem 1973, 16, 1200-1206. Meyers, A., Reuman, M. The Synthetic Utility of Oxazolines in Aromatic Substitution, Tetrahydron 1985, 41, 837-860. Novak, J., Salemink, A., Cannabis. Part 27.¹ Synthesis of 8-, 10-, and 11-Oxygenated Cannabinols, J Chem. Soc. Perkin Trans 1. 1983, 2867-2871. Hattori, T., Suzuki, T., and Miyano, S., A Practical and Efficient Method for the Construction of the Biphenyl Framework; Nucleophilic Aromatic Substitution on 2-Methoxybenzoates with Aryl Grignard Reagents, J. Chem. Soc., Chem. Commun. 1991, 1375-1376.

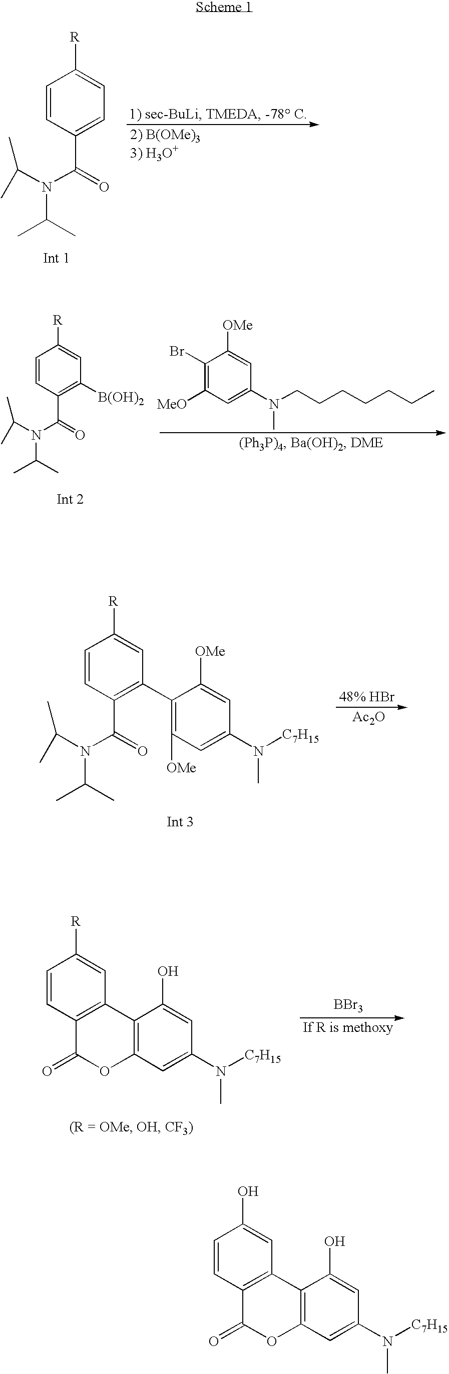

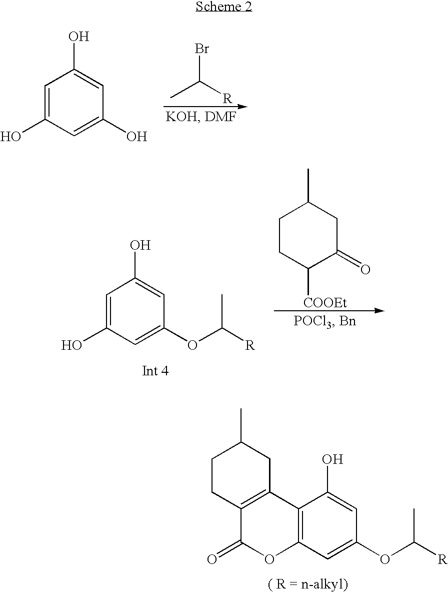

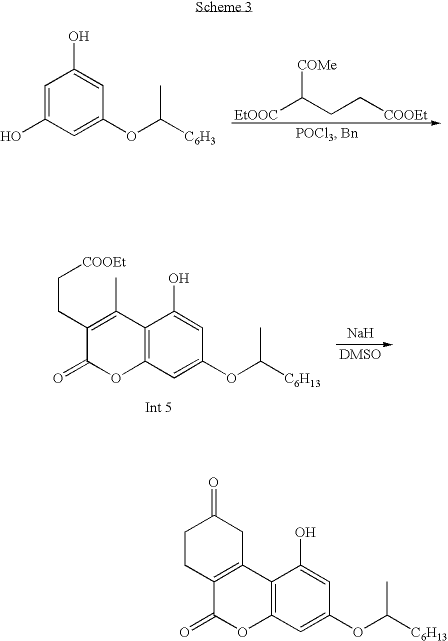

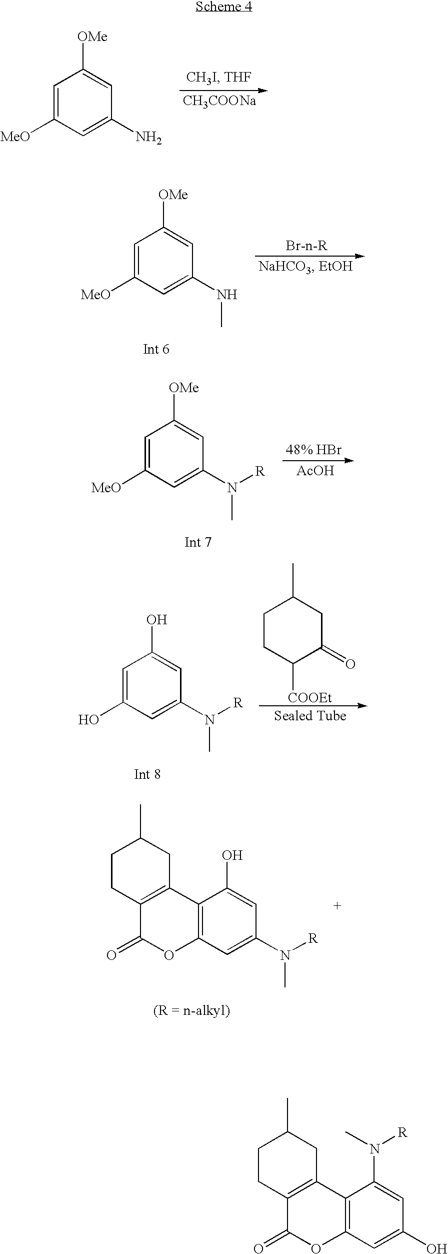

General Procedure for Synthesis of Compounds Prepared by Scheme 1

2-(N,N-Diisopropylcarboxamido)-5-alkyl/alkoxy/trifloromethyl-phenylboronic acid (Int 2). Anhydrous tetrahydrofuran (250 ml) and TMEDA (9.05 ml, 60 mmol) was cooled to −78° C. under argon and, with stirring, 46.2 ml (60 mmol) of 1.3 M sec-butyllithium solution was added via syringe. The yellow solution was stirred at the same temperature for about 5 minutes and a solution of 5-substituted-N,N-diisopropylbenzamide Int 1 (50 mmol) in 50 ml of anhydrous THF was added dropwise. The reaction mixture was stirred at −78° C. for 1 hr and then 16.4 ml (150 mmol) of trimethylborate was added dropwise. The reaction mixture was allowed to warm to room temperature over night. The pH of the mixture was adjusted to about 6.5 by addition of 12% aqueous hydrochloric acid (about 50 ml) and concentrated by vacuum evaporation. The residue was extracted with dichloromethane. The combined organic extracts were dried over Na₂SO₄. Removal of solvent afforded a yellow or white form of solid product. The solid product was recrystallized in hot diethyl ether (general yield is about 95%).

N,N-Diisopropyl-5-methyllmethoxy/trifloromethyl-1′6′dimethoxy4′-(-(N-methyl, N-heptyl)-2-biphenylcarboxamide (Int 3). The above boronic acid (8 mmol), tetrakistriphenylphosphine palladium (0.5 mmol) and barium hydroxide octahydrate (10 mmol) and dimethoxyethane (20 ml) were mixed; 4 ml of water was added and the reaction mixture was stirred at room temperature under argon for 10 min. Then, a solution of 4 mmol of the (4-Bromo-3,5-dimethoxy-phenyl)-heptyl-methyl-amine* in 10 ml of dimethoxyethane was added with stirring and the reaction mixture was stirred and refluxed under argon for 24 hr. After cooling to room temperature, the catalyst was filtered out with the facilitation of celite and the filtrate was concentrated by vacuum evaporation. The residue was chromatographed on silica gel (25% ethyl acetate in petroleum ether) to provide the product in a general yield of 50%.

*Note: (4-Bromo-3,5-dimethoxy-phenyl)-heptyl-methyl-amine was prepared via bromination of Int 7: To a solution of Int 7 (800 mg, 3 mmol), tetraethylammonium chloride mono hydrate (TEACI, 20 mg)), 60 microliter anhydrous methanol in 15 ml of anhydrous dichloromethane, 0.16 ml of bromine was added dropwise at 0° C. The reaction was quenched by addition of 50 ml of 10% sodium bicarbonate aqueous solution after stirring at 0° C. under argon atmosphere for 1 hr. The organic layer was separated and washed quickly with water, brine, and dried over Na₂SO₄ under the protection of argon. Filtration and removal of solvent afforded the 0.97 g of the title compound, which was used directly in the reaction without further purification.

3-(N-methyl, N-heptyl)-1-hydroxy-9-methyl/methoxy/hydroxy/trifloromethyl-6H-dibenzo-[b,d]-pyran-6-one (13, 18, 19). 10 ml of 48% hydrobromic acid was added dropwise to the solution of 2 mmol of Int 3 in 10 ml of acetic anhydride at 0° C., then the reaction mixture was stirred and heated at 90° C. for 24 hr. To obtain the 5-hydroxy product, the reaction needs to carried out for 8 more hr and the addition of 10 ml of 48% hydrobromic acid after 24 hr. The reaction mixture was then cooled to room temperature and treated with 20% NaOH aqueous solution to pH about 8.5. The mixture was then extracted with ether. The ethereal solution was separated and washed with water, brine and dried over Na₂SO₄. Filtration and removal of solvent provided yellow solid crude product. The crude was chromatographed on silica gel (40% dichloromethane-petroleum ether) to afford the expected product in a general yield of 60%.

General Procedure for Synthesis of Compounds Prepared by Scheme 2

5-(1-Methylalkyloxy)resorcinol (Int 4). To a solution of phloroglucinol (100 mmol) and potassium hydroxide (34 mmol) in 40 ml of anhydrous DMF was added 2-bromo-n-alkane (105 mmol). After stirring and heating the mixture for 16 hr at 100° C., the reaction mixture was cooled to room temperature and then treated with 25 ml of acetic acid. The reaction mixture was then filtered, and the filtrate was extracted with ethyl ether following addition of 200 ml of water, the ethereal solution was separated and washed with water thoroughly. The ethereal solution was extracted with 15% sodium hydroxide aqueous solution. The alkaline solution was separated and washed with ether, acidified with 2N HCl, and extracted with ethyl ether. The ethereal extraction was separated and washed with water, brine, and dried with Na₂SO₄. Filtration and removal of solvent provided a crude brownish product. The crude product was chromatographed on silica gel (35% acetone-petroleum ether) to afford the expected product in a general yield of 30%.

1-Hydroxy-9-methyl-3-(1-methyl-alkyloxy)-7,8,9,1 0-tetrahydro-benzo [c]chromen-6-one (2 & 3). To a stirred solution of the resorcinol Int 4 (2 mmol) and 4-Methyl-2-oxo-cyclohexanecarboxylic acid ethyl ester (2.1 mmol) in 50 ml of anhydrous benzene was added POCI₃ (2.1 mmol) dropwise. After stirring for 2 hr at room temperature, the reaction mixture was refluxed for 20 min and allowed to stir at room temperature over night. The mixture was then treated with water and refluxed for 15 min, after which ethyl ether was added. The ethereal phase was then separated and washed with 10% NaHCO₃ aqueous solution, water, brine, and dried (Na₂SO₄). Filtration and removal of solvent provided yellow solid crude product. The crude product was chromatographed on silica gel (25% acetone-petroleum ether) to afford the expected product in a general yield of 70%.

General Procedure for Synthesis of Compounds Prepared by Scheme 3

3-[5-Hydroxy-4-methyl-7-(1-methyl-heptyloxy)-2-oxo-2H-chromen-3-yl]-propionic acid ethyl ester (Int 5). Phosphorous oxychloride (0.78 ml, 8.4 mmol) was added to a solution of 5-(1-Methylheptyloxy)resorcinol Int 4 (2.0 g, 8.4 mmol) and diethyl 2-acetylglutarate (2.0 g, 8.4 mmol) in 15 ml of anhydrous benzene. The reaction mixture was then refluxed at 90° C. for 4 hr under argon atmosphere. The mixture was then cooled to room temperature and treated with 25 ml of water. Ethyl ether was added. The ethereal extraction was separated and washed with water, 5% NaHCO₃, brine, and dried over Na₂SO₄. Filtration and removal of solvent provided 3.28 g of a yellow oily crude product. The crude product was chromatographed on silica gel (25% acetone-petroleum ether) to afford the expected product 0.78 g (22% yield). mp 116-117° C. The product was characterized by GS-MS, and 1 D and 2D-¹H NMR.

1-Hydroxy-3-(1-methyl-heptyloxy)-7,10-dihydro-8H-benzo[c]chromere-6,9-dione (4). To a suspension of NaH (500 mg, 7.9 mmol, 60% dispersion in mineral oil, washed several times with n-hexane) in 4 ml of anhydrous DMSO, a solution of Int 5 (750 mg, 1.85 mmol) was added dropwise. The reaction mixture was stirred at room temperature over night. The reaction mixture was then poured onto ice containing 2 ml of 5% HCl. Ethyl ether was added to extract the solid crude product. The ethereal solution was separated and washed with water, brine, and dried over Na₂SO₄. Filtration and removal of solvent provided 640 mg yellow solid crude product. The crude product was chromatographed on silica gel (33% acetone-petroleum ether) to afford the expected product 340 mg (51% yield). mp 183-185° C. The product was characterized by GS-MS, and 1D-¹H NMR.

General Procedure for Synthesis of Compounds Prepared by Scheme 4

(3,5-Dimethoxy-phenyl)-methyl-amine (Int 6). A solution of 3,5-dimethoxyaniline (13.8 g, 90 mmol), lodomethane (12.7 g, 90 mmol), and sodium acetate (7.4 g, 90 mmol) in 100 ml of anhydrous THF was stirred under nitrogen atmosphere for 10 hr. Then, THF was removed by rotary evaporation. The crude product was partitioned between water and ethyl ether. The ethereal extraction was separated and washed with water, brine, and dried over Na₂SO₄. Filtration and removal of solvent provided 13.6 g of crude product. The crude product was chromatographed on silica gel (33% acetone-petroleum ether) to afford the expected product 4.5 g (27% yield).

(3,5-Dimethoxy-phenyl)-heptyl-methyl-amine (Int 7). A solution of Int 6 (5 g, 30 mmol), n-bromo-heptane (18.6 ml, 150 mmol), sodium hydrogen carbonate (3 g, 35 mmol) in 80 ml of anhydrous ethanol was stirred and heated at 90° C. for 16 hr. After cooling to room temperature, the solvent was removed by rotary evaporation. The crude product was partitioned between water and ethyl ether. The ethereal extraction was separated and washed with water, brine, and dried over Na₂SO₄. Filtration and removal of solvent provided 7.6 g of crude product. The crude product was chromatographed on silica gel (15% acetone-petroleum ether) to afford the expected product 4.8 g (64% yield). Another intermediate, Int 7 (3,5-dimethoxy-phenyl) -alkyl-methyl-amine), was prepared via the same procedure.

5-(Heptyl-methyl-amino)-resorcinol (Int 8). A mixture of Int 7 (570 mg, 2.3 mmol), 48% hydrobromic acid (13.5 ml) and glacial acetic acid (13.5 ml) was heated for 2 hr under argon atmosphere. The reaction mixture was then cooled to room temperature and treated with concentrated sodium hydroxide aqueous solution to pH 6.5. The resultant mixture was then extracted with ethyl ether. The ethereal extraction was separated and washed with water, brine, and dried over Na₂SO₄. Filtration and removal of solvent provided 550 mg of crude product. The crude product was chromatographed on silica gel (30% acetone-petroleum ether) to afford the expected product 500 mg (98% yield). Another intermediate, Int 8 (5-(alkyl-methyl-amino)-resorcinol), was prepared via the same procedure.

3-(Alkyl-methyl-amino)-1-hydroxy-9-methyl-7,8,9,10,-tetrahydro-benzo[c]chromen-6-one (9, 10, 11, 12). A solution of 2.0 mmol of 5-(N-methyl, N-alkyl amino)-resorcinol (Int 8) and 2.2 mmol of 4-Methyl-2-oxo-cyclohexanecarboxylic acid ethyl ester in 4 ml of anhydrous chloroform was stirred and heated at 110° C. under argon in a sealed tube for 16 hr. The reaction mixture was then cooled to room temperature. Solvent was removed by rotary evaporation. The resultant residue was chromatographed on silica gel (15% acetone-petroleum ether) to provide the two fluorescent products in a overall yield of 54%. The two isomers were characterized by GS-MS, and 1D and 2D-¹H NMR, the major product, 3-(alkyl-methyl-amino)-1-hyd roxy-9-methyl-7,8,9,10,-tetrahydro-benzo[c]chromen-6-one (Int 9 or Int 10), was collected in a general yield of 35%. 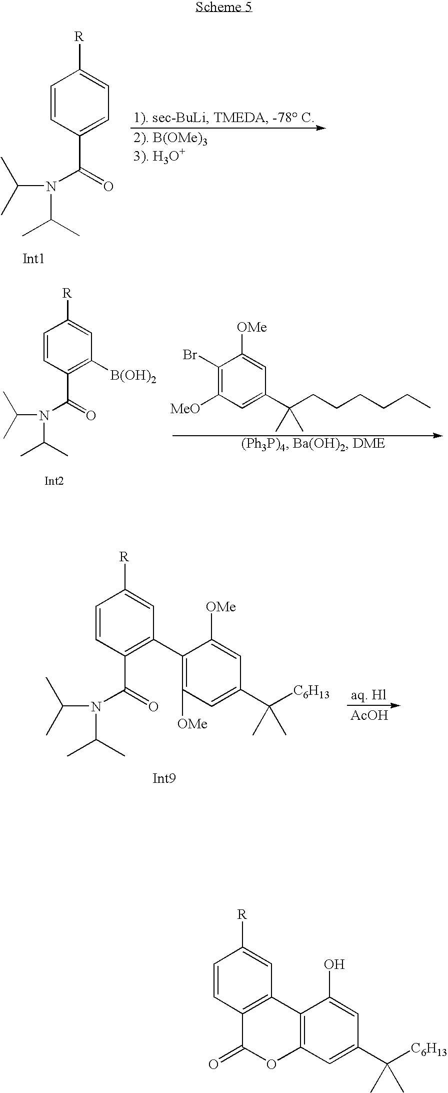 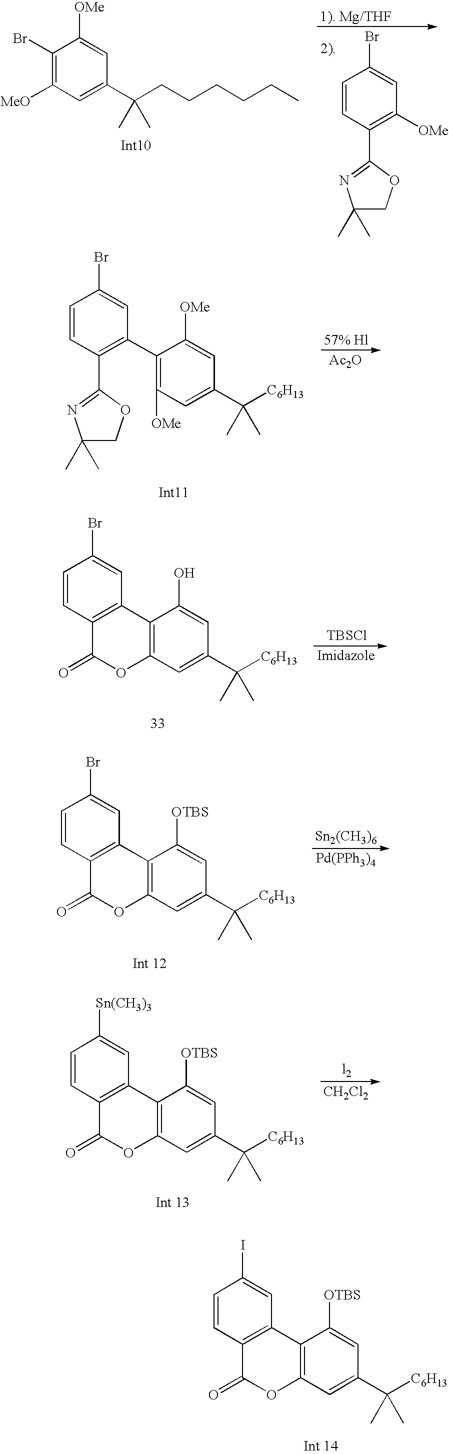
Synthesis of the compounds represented by Scheme 5 follows the similar method described in the procedures of Scheme 1. The synthesis of compounds and intermediates represented by Scheme 6 were carried out by an oxazoline facilitated aromatic carbon-carbon coupling reaction as described by Meyers, A., Reuman, M in “The Synthetic Utility of Oxazolines in Aromatic Substitution, Tetrahydron 1985, 41, 837-860”, and Novak, J., Salemink, A. in the “Cannabis. Part 27.¹ Synthesis of 8-, 10-, and 11-Oxygenated Cannabinols, J. Chem. Soc. Perkin Trans 1. 1983, 2867-2871”. The intermediates Int 12-Int 14 were further used to prepare the final products. Preparation of some of the final products were exemplified by Scheme 7, Scheme 8 and Scheme 9. 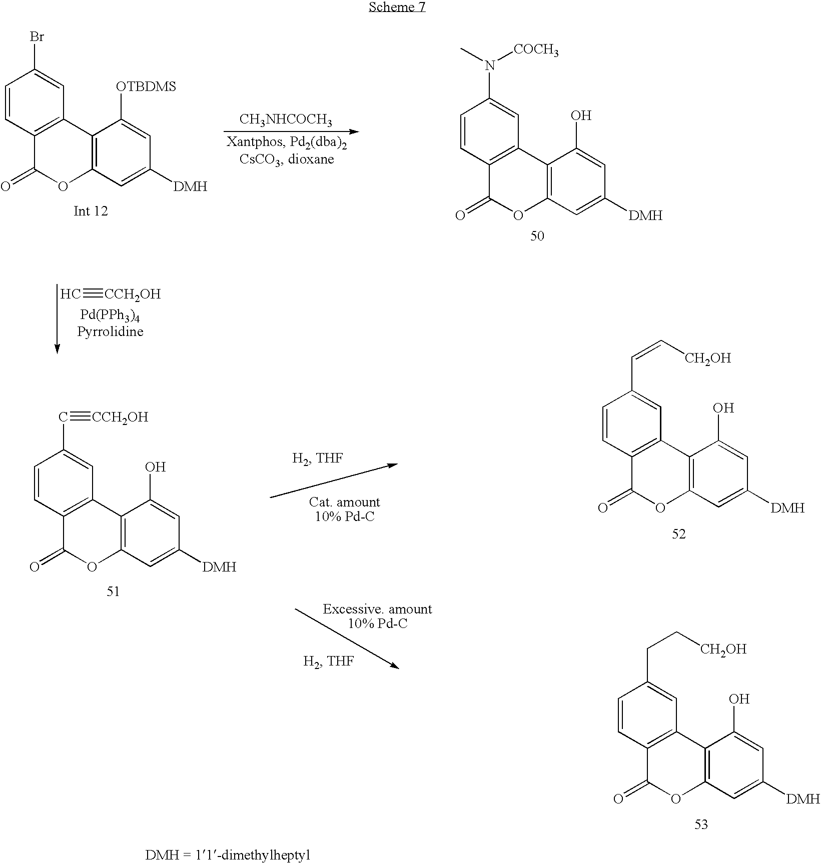 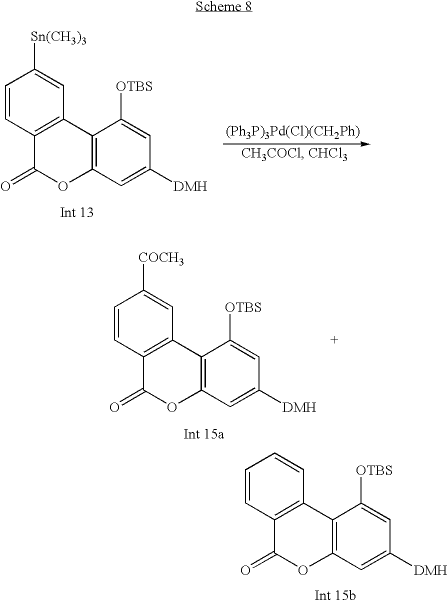 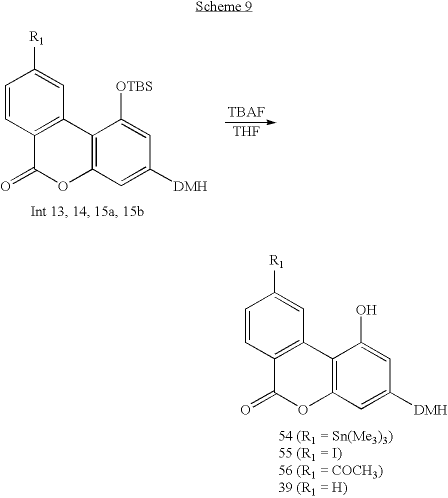
Preparation of the 2-bromo-5-(1′, 1′-dimethylheptyl)-1,3-dimethoxy-benzene (Int 10) was carried out according to the procedure described by Srebnik etc. in “Halogenation of phenols and phenyl ethers with potassium halides in the presence of 18-crown-6 on oxidation with m-chloroperbenzoic acid. J. Chem. Soc. Perkin. Trans. I EN 1987, 1423-1427”.

2-[4-Bromo-2-(2,6-dimethoxy-4-((1,1-dimethyl-heptyl)phenyl)phenyl]-4,4-dimethyl-4,5-dihydro-oxazole (Int 11). A portion of one forth of the solution of 4.95 g (14.4 mmol) of 2-Bromo-5-(1,1-dimethyl-heptyl)-1,3-dimethoxy-benzene (Int 10) in 25 mL of anhydrous THF was added under argon atmosphere to the flask containing 362 mg (15 mmol) of magnesium chip to initiate the formation of the Grignard reagent. The reset was added dropwise till completion of the addition. The mixture was refluxed for 1.5 h and then transferred to a flask containing a solution of the 2-(4-Bromo-2-methoxy-phenyl)-4,4-dimethyl-4,5-dihydro-oxazole. The resulted mixture was refluxed for 16 h under argon atmosphere, and then cooled to room temperature. The reaction was quenched by addition of 10 mL of saturated NH₄Cl aqueous solution. THF was removed by evaporation in reduced pressure. Ether was added to the residue to extract the product. The ethereal solution was washed with water and brine and dried with Na₂SO₄. Removal of solvent provided 5.75 g of liquid crude product, which was chomatographed on silica gel column eluted with 30% of ethyl acetate in petroleum ether to consecutively afford 2.0 g of 5-(1,1-dimethylheptyl)-1,3-dimethoxybenzene (recovered from the two equivalent Grignard reagent) and 3.234 g of the biphenyl oxazole Int 11 in a yield of 86.8% as a liquid. ¹H NMR (CDCl₃) δ 7.72 (d, J=8.1 Hz, 1H), 7.45 (d, J=2.1 Hz), 7.35 (dd, J=21. Hz, 8.1 Hz, 1H), 3.70 (s, 6H), 3.68 (s, 2H), 1.62-1.59 (m, 2H), 1.32 (s, 6H), 1.28-1.21 (m, 6H), 1.19 (s, 6H), 1.11-1.05 (m, 2H), 0.86 (t, J=7.1 Hz, 3H); MS m/z 515 (M⁺), 517, 484 (100%), 486.

9-Bromo-3-(1,1-dimethyl-heptyl)-1-hydroxy-benzo[c]chomen-6-one (33). The solution of the biphenyl oxazoline Int 11 (3.20 g , 6.21 mmol) in 21 mL of acetic anhydride was carefully added 21 mL of 57% hydriodic acid dropwise at room temperature. The mixture was stirred and refluxed for 24 h under argon atmosphere. The mixture was cooled to room temperature and treated with 3N NaOH aqueous solution to adjust the solution pH to 7.5-8. Ether was added to extract the product. The ethereal solution was separated and washed with 10% sodium hydrogen sulfate solution, water and brine, and dried. Removal of solvent provided 2.41 g of white solid, which was recrystallized in acetone to afford 2.25 g of 33 as white crystalline (86.6%). Mp. 230-231° C.; ¹H NMR (CDCl₃) δ 9.19 (d, J=1.8 Hz, 1H), 8.26 (d, J=8.5 Hz, 1H), 7.65 (dd, J=8.5 Hz, 1.8 Hz, 1H), 6.95 (d, J=1.7 Hz, 1H), 6.73 (d, J=1.7 Hz, 1H), 6.14 (s, 1H, OH), 1.60-1.51 (m, 2H), 1.29 (s, 6H), 1.24-1.18 (m, 6H), 1.06-1.03 (m, 2H), 0.83 (t, J=7.1 Hz, 3H); MS m/z 416 (M⁺), 418; Anal. (C₂₃H₂₅BrO₃.1/4H₂O), C, H, calculated, C 62.63%, H 5.99%, found, C 62.82%, H 5.71%.

9-Bromo-1-(tert-butyl-dimethyl-silanyloxy)-3-(1,1-dimethyl-heptyl)-benzo [c]chomen-6-one (Int 12). The mixture of compound 33 (1.41 g, 3.38 mmol), tert-butyldimethylsilyl chloride (550 mg, 3.54 mmol) and imidazole (460 mg, 6.78 mmol) was stirred at room temperature under argon atmosphere for 50 h. The reaction was then quenched by addition of 100 mL of water. The organic phase was separated and washed with water and brine and dried. Removal of solvent provided 2.26 g of light yellow liquid, which was chomatographed on silica gel column eluted with 2.5% acetone in petroleum ether to afford 1.74 g of Int 12 in a yield of 97.1% as a white solid. Mp 49-50° C.; ¹H NMR (CDCl₃) δ 9.25 (d, J=1.8 Hz, 1H), 8.24 (d, J=8.45 Hz, 1H), 7.62 (dd, J=1.8 Hz, 8.45 Hz, 1H), 6.96 (d, J=1.8 Hz, 1H), 6.8 (d, J=1.8 Hz, 1H), 1.61-1.57 (m, 2H), 1.29 (s, 6H), 1.23-1.20 (m, 6H), 1.08-1.03 (m, 11H, especially, 1.08, s, 9H), 0.84 (t, J=7.1 Hz, 3H), 0.41 (s, 6H); MS m/z 530 (M⁺), 532.

1-(tert-Butyl-dimethyl-silanyloxy)-3-(1,1-dimethyl-heptyl)-9-trimethylstannanyl-benzo [c]chomen-6-one (Int 13). The solution of Int 12 (532 mg, 1 mmol) and hexamethylditin (490 mg, 1.5 mmol) in 8 mL of anhydrous toluene was bubbled by argon flow for 10 min, then Pd(PPh₃)₄ (231 mg, 0.2 mmol) was added. The mixture saturated with argon for 5 min and then stirred and heated in pressure tube at 100° C. for 12 h. Reaction mixture was cooled to room temperature and filtered with a short silica gel column. The filtrate was concentrated under vacuum to afford 872 mg of liquid crude product, which was chomatographed on silica gel column eluted with 6% of diethyl ether in petroleum ether to provide 547 mg of Int 13 in a yield of 88.9% as a white solid. Mp 62-63° C.; ¹H NMR (CDC1₃) δ 9.25 (a triplet like AB system from Sn—H coupling, J=23.1 Hz, 1H), 8.32 {m,1H, especially, doublet of a triplet like AB system from Sn—H coupling, J=7.6 Hz (d), J=5.1 Hz (Sn—H)}, 7.65 (m, 1H, especially, double doublet of a triplet like AB system from Sn-H coupling, J=20.0 Hz (Sn—H), J=7.6 Hz (d), J=0.7 Hz (d)}, 6.97 (d, J=1.9 Hz, ), 6.81 (d, J=1.9 Hz, 1H), 1.61-1.57 (m, 2H), 1.29 (s, 6H), 1.25-1.18 (m, 6H), 1.10-1.00 m, 11H, especially, 1.01, s, 9H), 0.83 (t, J=7.1 Hz, 3H), 0.45-0.31 {15 H, especially, 0.383 (s, 6H, SiMe₂), and 0.379 (9H, 2 sets of AB, which could be from ¹¹⁷Sn—H and ¹¹9Sn—H coupling, J=27.72 Hz and J=27.99 Hz}; MS m/z 599 (M⁺—CH₄), 559, 531, 475, 395, 371, 351, 309, 165 (100%).

1-(tert-Butyl-dimethyl-silanyloxy)-3-(1,1-dimethyl-heptyl)-9-iodo-benzo [c]chomen-6-one (Int 14). The solution of Int 13 (138 mg, 0.22 mmol) in 10 mL of anhydrous dichloromethane was added a solution of iodine (77.4 mg, 0.3 mmol) in 5 mL of anhydrous dichloromethane. The mixture was stirred for 15 min at room temperature. The reaction was quenched by addition of 8 mL of sodium hydrogen sulfite aqueous solution. The organic phase was separated and washed with water and brine and dried. Removal of solvent provided 131 mg of liquid crude product, which was purified on silica gel column eluted with 6% of diethyl ether in petroleum ether to afford 116 mg of Int 14 in a yield of 83.4% as colorless oil. ¹H NMR (CDCl₃) δ 9.45 (d, J=1.6 Hz, 1H), 8.07 (d, J=8.3 Hz, 1H), 7.85 (dd, J=8.3 Hz, 1.6 Hz, 1H), 6.96 (d, J=1.8 Hz, 1H), 6.80 (d, J=1.8 Hz, 1 H), 1.61-1.56 (m, 2H), 1.29 (s, 6H), 1.25-1.18 (m, 6H), 1.08 (s, 9H), 1.07-1.02 (m, 2H), 0.84 (t, J =7.1 Hz, 3H), 0.42 (s, 6H); MS m/z 578 (M⁺).

N-[3-(1,1-Dimethyl-heptyl)-1-hydroxy-6-oxo-6H-benzo[c]chomen-9-yl]-N-methyl-acetamide (50). Argon was bubbled for 10 min though the mixture of Int 12 (133 mg, 0.25 mmol), N-methylacetamide (28 mg, 0.38 mmol), xantphos (18 mg, 0.03 mmol) and cesium carbonate (128 mg, 0.39 mmol) in 1.5 mL of anhydrous 1,4-dioxane in a pressure tube capped with a septum. Then, 10 mg (0.011 mmol) of Pd₂(dba)₃ (tris(dibenzilideneacetone)dipalladium(0)) was added to the mixture and the argon bubble flow was continued for 5 more min. The septum was replaced quickly with Teflon cap. The pressure tube was flashed with argon before the cap was tightly sealed. The reaction mixture was stirred and heated at 100° C. for 20 h and then cooled to room temperature, and was added 8 mL of dichloromethane. The reaction mixture was filtered by a short silica gel column. The filtrate was concentrated by vacuum evaporation to afford 123 mg of solid crude product. The crude product was chomatographed on silica gel column eluted with 30% acetone in petroleum ether to afford 50 in a yield of 60.0% as a white solid (R_{f}=0.4, 30% acetone in petroleum ether). Mp 81-83° C.; ¹H NMR (CDCl₃) δ 9.32 (bs, 1H, OH)), 9.05 (d, J=2.0 Hz, 1H), 8.49 (d, J=8.5 Hz, 1 H), 7.37 (dd, J=8.5 Hz, 2.0 Hz, 1H), 6.97 (bs, 1H), 6.93 (d, J=2.0 Hz, 1H), 3.46 (s, 3H), 2.11 (bs, 3H), 1.63-1.60 (m, 2H), 1.32 (s, 6H), 1.25-1.15 (m, 6H), 1.10-1.05 (m, 2H), 0.83 (t, J=7.0 Hz, 3H); NOE correlations: OH with H-2, N-methyl with H-8 and H-10; MS m/z 409 (M⁺), 395, 338, 253 (100%); Anal. (C₂₅H₃₁NO₄), C, H, N; calculated C 73.32%, H 7.63%, N 3.42%, found, C 73.36%, H 7.90%, N 3.15%.

3-(1,1-Dimethyl-heptyl)-1-hydroxy-9-(3-hydroxy-prop-1-ynyl)-benzo[c]chomen-6-one (51). Argon was bubbled though the mixture of Int 12 (100 mg, 0.188 mmol), propargyl alcohol (30 mg, 0.54 mmol) in 4 mL of redistilled pyrrolidine for 10 min. 10 mg of Pd(PPh₃)₄ was added to the mixture. The argon bubble flow was continued for 5 more min. The reaction mixture was stirred and heated at 70° C. for 7 h under argon atmosphere. The reaction mixture was cooled to room temperature and treated with 10 mL of saturated NH₄Cl aqueous solution. Ethyl acetate was added to extract the product. The organic layer was separated and washed with water and brine and dried. Removal of solvent provided a solid crude, which was chomatographed on silica gel column eluted with 20% of ethyl acetate in petroleum ether to afford 56 mg of final product 51 in a yield of 76% as a white solid. Mp 215-217° C.; ¹H NMR (CD₃COCD₃) δ 9.97 (bs, 1H, OH), 9.20 (d, J=1.5 Hz, 1H), 8.30 (d, J=8.1 Hz, 1H), 7.59 (dd, J=1.5 and 8.1 Hz, 1H), 6.98 (d, J=2.0 Hz, 1H), 6.88 (d, J=2.0 Hz, 1H), 4.49 (bs, 2H), 4.27 (bs, 1H, OH), 1.67-1.64 (m, 2H), 1.32 (s, 6H), 1.27-1.17 (m, 6H), 1.14-1.06 (m, 2H), 0.83 (t, J=6.9 Hz, 3H); MS m/z 392 (M⁺); Anal. (C₂₅H₂₈O₄.3/4H₂O), C, H, calculated C 73.98%, H 7.27%, found, C 74.27%, H 7.36%.

3-(1,1-Dimethyl-heptyl)-1-hydroxy-9-(3-hydroxy-cis-propenyl)-benzo[c]chomen-6-one (52). The mixture of 22 mg (0.056 mmol) of 51 and 7 mg of 10% Pd—C in 4 mL of anhydrous THF was stirred at room temperature under hydrogen atmosphere for 3 h. The mixture was then diluted with dichloromethane and filtered. The filtrate was concentrated and chomatographed on silica gel column eluted with 30% ethyl acetate in petroleum ether to afford 15.8 mg of a mixture of ZIE isomer (ratio: 95:5 by ¹H NMR) in a total yield of 71.6% as a white solid. The solid was recrystallized in acetone and provided 11.8 mg of pure Z isomer 52. Mp 138-139° C.; ¹H NMR (CD₃COCD₃) δ 9.09 (d, J=1.5 Hz, 1H), 8.28 (d, J=8.1 Hz, 1H), 7.47 (dd, J=1.5 Hz, 8.1 Hz, 1 H), 7.02 (d, J=1.9 Hz, 1 H), 6.84 (d, J=1.9 Hz, 1 H), 6.63 (dt, J =12.0 Hz, 2.0 Hz, 1H), 6.07 (dt, J=12.0 Hz, 5.9 Hz, 1H), 4.53 (dd, J=5.9 Hz, 2.0 Hz, 2H), 2.95 (bs, 1H), 1.65-1.62 (m, 2H), 1.31 (s, 6H), 1.24-1.17 (m, 6H), 1.11-1.08 (m, 2H), 0.82 (t, J=6.9 Hz, 3H); MS m/z 394 (M⁺); Anal. (C₂₅H₃₀O₄.H₂O), C, H, calculated C 72.72%, H 7.75%, found, C 72.43%, H 7.40%.

3-(1,1-Dimethyl-heptyl)-1-hydroxy-9-(3-hydroxy-propyl)-benzo[c]chomen-6-one (53). The mixture of 30 mg (0.076 mmol) of 51 and 18 mg of 10% Pd—C in 5 mL of anhydrous THF was stirred at room temperature under hydrogen atmosphere for 2 h. The mixture was then diluted with dichloromethane and filtered. The filtrate was concentrated and chomatographed on silica gel column eluted with 40% ethyl acetate in petroleum ether to afford 22 mg of 53 in a yield of 73.1% as a white solid. Mp 48-50° C.; ¹H NMR (CD₃COCD₃) δ 8.92 (s, 1H), 8.30 (d, J=8.0 Hz, 1H), 7.74 (bs, 1H, OH), 7.32 (dd, J=1.5 Hz, 8.0 Hz, 1H), 6.89 (d, J=1.5 Hz, 1H), 6.80 (d, J=1.5 Hz, 1H), 3.77 (t, J=6.5 Hz, 2H), 2.89 (t, J=7.5 Hz, 2H), 2.35 (bs, 1H), 2.04-1.98 (m, 2H), 1.56-1.52 (m, 2H), 1.25 (s, 6H), 1.20-1.11 (m, 6H), 1.05-0.98 (m, 2H), 0.79 (t, J =7.0 Hz); MS m/z 396 (M⁺); Anal. (C₂₅H₃₂O₄)

9-Acetyl-1-(tert-butyl-dimethyl-silanyloxy)-3-(1,1-dimethyl-heptyl)-benzo [c]chomen-6-one (Int 15a) and 1-(tert-Butyl-dimethyl-silanyloxy)-3-(1,1-dimethyl-heptyl)-benzo[c]chomen-6-one (Int 15b). Argon was bubbled for 10 min though the mixture of Int 13 (100mg, 0.16 mmol), acetyl chloride (13 mg, 0.165 mmol) in 3 mL of anhydrous chloroform in a pressure tube capped with a septum. Benzyl-bis(triphenylphosphine)palladium(ll) chloride (10 mg, 0.013 mmol) was then added to the mixture and the argon bubble flow was continued for 5 more min. The septum was replaced quickly with Teflon cap. The pressure tube was flashed with argon before the cap was tightly sealed. The reaction mixture was stirred and heated at 70° C. for 20 h and then cooled to room temperature, and dichloromethane was added. The resulted mixture was stirred for 40 min and then filtered with s short silica gel column. The filtrate was washed with 10% sodium bicarbonate solution, water and brine, and dried. Removal of solvent provided 82 mg of liquid crude product. The crude product was chomatographed on silica gel column eluted with 10% acetone in petroleum ether provided two viscous oil products The first fraction (R_{f}=0.8) afforded 37mg of 15b in a yield of 51.1%. The second fraction (R_{f}=0.5) gave 22 mg 15a in a yield of 27.8%. 15a: ¹H NMR (CDCl₃) δ 9.55 (d, J=1.0 Hz, 1H), 8.50 (d, J=8.0 Hz, 1H), 8.01 (d, J=1.0 Hz, 8.0 Hz, 1H), 6.98 (d, J=1.5 Hz, 1H), 6.86 (d, J=1.5 Hz, 1H), 2.72 (s, 3H), 1.62-1.58 (m, 2H), 1.30 (s, 6H), 1.25-1.19 (m, 6H), 1.06-1.03 (m, 11H, especially, 1.03, s, 9H), 0.84 (t, J=7.0 Hz, 3H), 0.47 (s, 6H); MS m/z 494 (M⁺). 15b: ¹H NMR (CDCl₃) 6 9.02 (dd, J=8.3 Hz, 1.0 Hz, 1H), 8.41 (dd, J=8.0 Hz, 1.5 Hz, 1H), 7.74 (dt, J=1.5 Hz, 8.3 Hz, 1H), 7.51(dt, J=1.0 Hz, 8.0 Hz, 1H), 6.98 (d, J =1.5 Hz, 1H), 6.80 (d, J=1.5 Hz, 1H), 1.61-1.58 (m, 2H), 1.29 (s, 6H), 1.25-1.6 (m, 6H), 1.10-1.03 (m, 11H, especially, 1.06, s, 9H), 0.84 (t, J=6.7 Hz, 3H), 0.37 (s, 6H); MS m/z 452 (M⁺), 395 (100%).

General procedure for removal of t-butyidimethylsilyl (TBS) protective group by tetrabutyl ammonium fluoride (TBAF). A solution of 1-butyidimethylsilyl-cannabinolactone (1.0 mmol) in THF (10 mL) was added TBAF solution (1.25 mL, 1.0 M in THF) dropwise at room temperature. The reaction mixture was stirred for 10-15 min and was treated with 10 mL of saturated NH₄Cl aqueous solution. THF was removed by vacuum evaporation. The residue was extracted with ethyl acetate (50 mL). The organic phase was separated and washed with water and brine and dried with anhydrous Na₂SO₄. Filtration and removal of solvent provided crude product in a solid form. The crude was chomatographed on silica gel column eluted with 25-30% of acetone in petroleum ether to afford the product.

3-(1,1-Dimethyl-heptyl)-1-hydroxy-9-trimethylstannanyl-benzo[c]chomen-6-one (54). 33 mg of 54 was prepared from 45 mg of Int 13 following the general procedure of removal of silyl protective group in a yield of 90.2% as a white solid. Mp 160-161° C.; ¹H NMR (CDCl₃) δ 9.17 (1H, a triplet like AB system from Sn—H coupling, J=24.0 Hz), 8.34{1H, doublet of a triplet like AB system from Sn—H coupling, J=7.5 Hz (d), J=5.0 Hz (t)}, 7.67 {m, 1H, especially, double doublet of a triplet like AB system from Sn—H coupling, J=0.5 Hz (d), 7.5 Hz (d), J=19.5 Hz (t, Sn—H)}, 6.07 (s, 1H, OH), 1.60-1.57 (m, 2H), 1.29 (s, 6H), 1.24-1.15 (m, 6H), 1.08-1.02 (m, 2H), 0.83 (t, J=7.0 Hz, 3H), 0.51-0.24 {m, 9H, especially, 3 sets of a triplet like AB system from ¹¹⁵Sn—H coupling, ¹¹⁷Sn—H coupling and ¹¹⁹Sn—H coupling with center peak at 0.38, J=63.5 Hz, J=27.5 Hz, J=26.5 Hz); MS m/z 502 (M⁺), 338, 296, 282, 268, 253 (100%), 239, 225, 213, 197. Anal. (C₂₅H₃₄O₃Sn), C, H; calculated C 59.90%, H 6.84%, found, 59.60%, H 7.01%.

3-(1,1-Dimethyl-heptyl)-1-hydroxy-9-iodo-benzo[c]chomen-6-one (55). 61 mg of 55 was prepared from 95 mg of Int 14 following the general procedure of removal of silyl protective group in a yield of 80.3% as a white solid. Mp 234-235° C.; ¹H NMR (CD₃COCD₃) δ 9.82 (bs, 1 H), 9.57 (d, J=1.6 Hz, 1 H), 8.04 (d, J=8.3 Hz, 1 H), 7.97 (dd, J=1.6 Hz, 8.3 Hz, 1 H), 6.99 (d, J=1.9 Hz, 1H), 6.89 (d, J=1.9 Hz, 1H), 1.67-1.63 (m, 2H), 1.32 (s, 6H), 1.26-1.18 (m, 6H), 1.13-1.07 (m, 2H), 0.82 (t, J =7.1 Hz, 3H); MS m/z 464 (M⁺); Anal. (C₂₂H₂₅IO₃), C, H; calculated C 56.91%, H 5.43%, found, C 56.96%, H 5.47%.

9-Acetyl-3-(1,1-dimethyl-heptyl)-1-hydroxy-benzo[c]chomen-6-one (56). 13.1 mg of 56 was prepared from 18.0 mg of Int 15a following the general method of removal of silyl protective group in a yield of 95.7% as a white solid. Mp 177-178° C.; ¹H NMR (CDCl₃) δ 9.71 (d, J=1.5 Hz, 1H), 8.51 (d, J=8.5 Hz, 1H), 8.05 (dd, J=1.5 Hz, 8.5 Hz, 1H), 7.27 (bs, 1H, OH), 6.96 (d, J=2.0 Hz, 1H), 6.93 (d, J=2.0 Hz, 1H), 2.79 (s, 3H), 1.62-1.59 (m, 2H), 1.31 (s, 6H), 1.24-1.16 (m, 6H), 1.08-1.05 (m, 2H), 0.82 (t, J =7.0 Hz, 3H); MS m/z 380 (M⁺); Anal. (C₂₄H₂₈O₄), C, H, calculated C 75.76%, H 7.42%, found, C 75.56%, H 7.63%.

3-(1,1-Dimethyl-heptyl)-1-hydroxy-benzo[c]chomen-6-one (39). Following the general procedure of removal of silyl protective group, 30 mg of 39 was prepared from 50 mg of Int 15b in a yield of 83.7% as a white solid. Mp 170-171° C.; ¹H NMR (CDCl₃) δ 9.05 (dd, J=8.5 Hz, 1.0 Hz, 1H), 8.44 (dd, J=1.5 Hz, 8.0 Hz, 1H), 7.80 (dt, J=1.5 Hz, 8.5 Hz, 1H), 7.53 (dt, J=1.0 Hz, 8.0 Hz, 1H), 6.95 (d, J=1.5 Hz, 1H), 6.79 (d, J=1.5 Hz, 1H), 6.47 (s, 1H, OH), 1.60-1.57 (m, 2H), 1.29 (s, 6H), 1.24-1.16 (m, 6H), 1.08-1.04 (m, 2H), 0.82 (t, J=7.0 Hz, 3H); MS m/z 338 (M⁺); Anal. (C₂₂H₂₆O₃), C, H, calculated C 78.07%, H 7.74%, found, C, 78.14%; H, 7.69%.

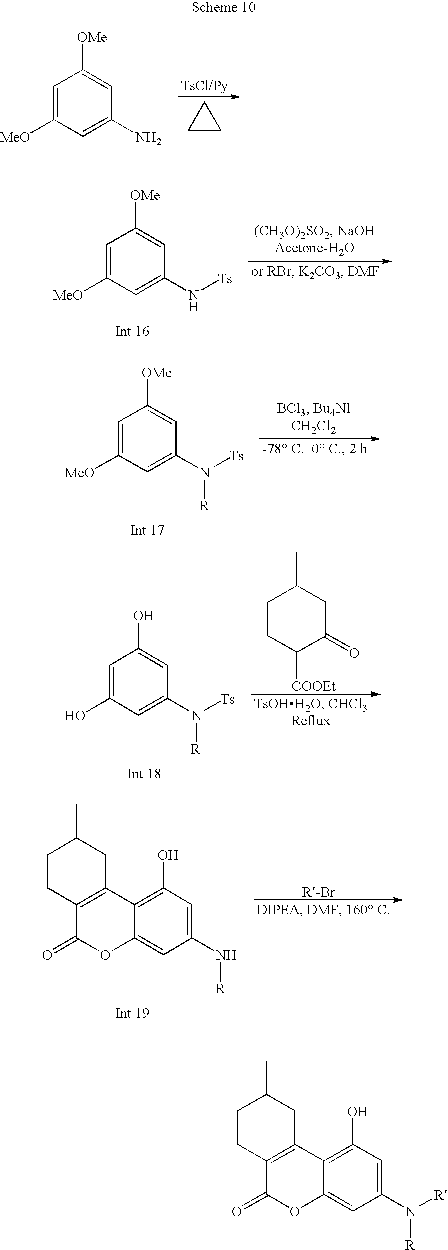

N-(3,5-Dimethoxy-phenyl)4-methyl-benzenesulfonamide (Int 16). The mixture of 3,5-dimethoxylaniline (24.0 g, 156.9 mmol) and p-toluenesulfonyl chloride (29.8 g, 156.9 mmol) in anhydrous pyridine (70 mL) was stirred and heated to reflux for 30 min under argon atmosphere. The reaction mixture was cooled to room temperature and poured into 400 mL of ice-cold water. Dichloromethane was added to extract the product. The organic layer was separated and washed with water, 5% HCl aqueous solution, water and brine and dried with anhydrous Na₂SO₄. Removal of solvent provided yellow solid crude, which was purified by re-crystallization in diethyl ether to afford 42.6 g (89.0%) of Int 16 as light yellow crystalline. mp 103-104° C.; ¹H NMR (CDCl₃) δ 7.74 (d, J=9.2 Hz, 2H), 7.26 (d, J=9.2 Hz, 2H), 6.94 (s, 1H), 6.26 (d, J=2.2 Hz, 2H), 6.17 (d, J=2.2 Hz, 1H), 3.70 (s, 6H), 2.38 (s, 3H); MS m/e 307 (M⁺).

N-(3,5-Dimethoxy-phenyl)-4, N-dimethyl-benzene-sulfonamide (Int 17a). A solution of the sulfonamide Int 14 (7.0 g, 22.8 mmol) and NaOH (1.58 g, 39.4 mmol) in a mixture of acetone-water (2:1, 150 mL) was added dimethylsulfate (3.5 mL, 37.1 mol) dropwise with stirring at room temperature. The reaction mixture was stirred at RT for 3 h and white crystalline formed. The white crystals were filtered out and washed with water, and dried in desiccator with phosphorus pentoxide under vacuum. After drying, 7.10 g (97.0%) white crystalline of Int 17a was collected. mp 110-111° C.; ¹H NMR (CDCl₃) δ 7.50 (d, J=8.0 Hz, 2H), 7.28 (d, J=8.0 Hz, 2H), 6.37 (d, J=1.85 Hz 1H), 6.27 (d, J=1.85 Hz, 2H), 3.73 (s, 6H), 3.13 (s, 3H), 2.42 (s, 3H), MS m/e 321 (M⁺).

N-(3,5-Dimethoxy-phenyl)-N-heptyl-4-methyl-benzenesulfonamide (Int 17b). The mixture of sulfonamide Int 16 (15.0 g, 48.9 mmol), 1-bromoheptane (9.63 g, 53.8 mmol) and potassium carbonate (17.1 g, 124.2 mmol) in anhydrous DMF (100 mL) was stirred and heated at 120° C. for 4 h, and then cooled to room temperature and poured into ice-cold water (400 mL). Diethyl ether (400 mL) was added to extract product. The ethereal solution was separated and washed with water, brine and dried with Na₂SO₄. Removal of solvent and drying reagent provided 19.50 g of light yellow liquid, which was treated with small amount of diethyl ether to give 19.10 g (96.4%) of Int 17b as white solid upon cooling. ¹H NMR (CDCl₃) δ 7.53 (d, J =8.1 Hz, 2H), 7.25 (d, J=8.1 Hz, 2H), 6.39 (t, J=2.1 Hz 1H), 6.20 (d, J=2.1 Hz, 2H), 3.71 (s, 6H), 3.44 (t, J=7.0 Hz, 2H), 2.42 (s, 3H), 1.43-1.40 (m, 2H), 1.29-1.21 (m, 8H), 0.85 (t, J=7.1 Hz, 3H); MS m/e 405 (M⁺).

N-(3,5-Dimethoxy-phenyl)-N-su bstituted-4-methyl-benzenesulfonamide (Int 17). Another variation of Int 17 by alkylation of Int 16 with substituted alkyl halide was carried out by following the procedure for preparation of Int 17b.

General procedure for preparation of Int 18. BCl₃ solution (145 mL, 1.0 M in CH₂Cl₂) was added dropwise to the solution of 17 (47 mmol) and tetrabutylammonium iodide (53.5 g, 145 mmol) in 250 mL of anhydrous dichloromethane at −78° C. under argon atmosphere. The reaction mixture was stirred at −78° C. for 20 minutes, and then at 0° C. for 2 h. Reaction was quenched by addition of 100 mL of water slowly. The organic layer was separated and washed with water, 30% aqueous NaHSO₃ solution, water and brine, and dried with anhydrous sodium sulfate. Removal of solvent and drying reagent provided semi-solid crude product. The crude product was chromatographed on silica gel column eluted with a mixture of petroleum ether and acetone (2:1) to afford Int 18 (90-95%) as a light yellow solid. 5-N-Methyl-N-tosyl-resorcinol (Int 18a): mp. 71-73° C.; ¹H NMR (CDCl₃) δ 7.51 (d, J=8.0 Hz, 2H), 7.24 (d, J=8.0 Hz, 2H), 6.32 (d, J=1.5 Hz 1H), 6.24 (d, J=1.5 Hz, 2H), 6.16 (bs, 2H), 3.09 (s, 3H), 2.40 (s, 3H), MS m/e 293 (M⁺). 5-N-Heptyl-N-tosyl-resorcinol (Int 18b): ¹H NMR (CDCl₃) δ 7.55 (d, J=8.1 Hz, 2H), 7.27 (d, J=8.1 Hz, 2H), 6.50 (bs, 2H), 6.35 (t, J=1.6 Hz 1H), 6.24 (d, J=1.6 Hz, 2H), 3.41 (t, J=7.2 Hz, 2H), 2.40 (s, 3H), 1.43-1.38 (m, 2H), 1.27-1.19 (m, 8H), 0.84 (t, J=7.0 Hz, 3H), MS m/e 377 (M⁺).

General procedure for preparation of Int 19. The mixture of Int 18 (5.0 mmol) and 4-Methyl-2-oxo-cyclohexanecarboxylic acid ethyl ester (6.0 mmol) and p-TsOH mono hydrate (7.5 mmol) in 100 mL of anhydrous CHCl₃ was refluxed for 108 hours under argon atmosphere. The reaction mixture was cooled to room temperature and treated with water (25 mL), followed by treatment of saturated NaHCO₃ aqueous solution to pH about 8.5. The organic layer was separated and washed with water and brine and dried. Removal of solvent and drying reagent (Na₂SO₄) provided brownish crude, which was chromatographed on silica gel column eluted with the mixture of petroleum ether-acetone (3:1) to afford the fluorescent product Int 19 as light yellow solid in general yield of 40%.

1-Hydroxy-9-methyl-3-methylamino-7,8,9,10-tetrahydro-benzo[c]chromen-6-one (Int 19a): ¹H NMR (CD₃COCD₃) δ 8.88 (s, 1H), 6.07 (d, J=2.0 Hz, 1 H), 5.98 (d, J=2.0 Hz, 1H), 5.54 (bs, 1H), 3.40 (dd, J=3.5 Hz, 19.5 Hz, 1H), 2.79 (s, 3H), 2.64-2.57 (m, 1H), 2.55-2.50 (m, 1H), 2.37-2.28 (m, 1H), 1.85-1.81 (m, 1H), 1.77-1.68 (m, 1H), 1.25 (td, J=12.5 Hz, 5.5 Hz, 1H), 1.07 (d, J=7.0 Hz, 3H) MS m/e 259 (M⁺).

General procedure for alkylation of Int 19. The mixture of Int 19 (1 mmol) and substituted alkyl halide (1.2 mmol) and Hunig's base (DIPEA) (1.2 mmol) in DMF (2˜3 mL) was heated and stirred in pressure tube at 160° C. for about 48 h. The reaction mixture was cooled to room temperature and treat with 60 mL of water and extracted with dichloromethane. The organic layer was separated and washed with water and 4% HCl, water, NaHCO₃ aqueous solution and brine, and dried. Removal of solvent and drying reagent (Na₂SO₄) provided brownish crude solid product, which was chromatographed on silica gel column eluted with the mixture of petroleum ether-acetone (4:1) to afford the fluorescent product as light yellow solid in general yield of 65%. 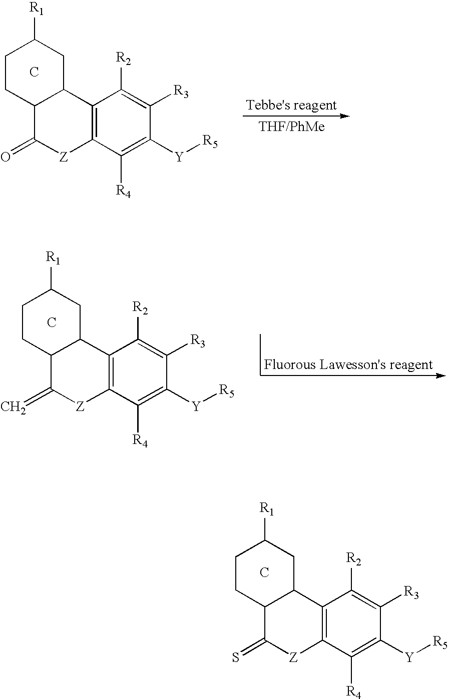
Some of the compounds disclosed in this invention are believed able to be converted into analogs wherein W is C═CH₂ or C═S as illustrated by Scheme 11 through application of Tebbe's reagent or Lawesson's reagent respectively. These are supported by the following reported methods: 1) as described by Zhi etc. in 5-benzylidene-1,2-dihydrochromeno[3,4-f]quinolines as selective progesterone receptor modulators, J. Med. Chem. 2003, 46, 4104-4112; and 2) as described by Kaleta etc. in Synthesis and Application of a Fluorous Lawesson's Reagent: Convenient Chromatography-Free Product Purification., Org. Lett., 2006, 1093-1095.

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed is:

1. A compound of formula I, and physiologically acceptable salts thereof, 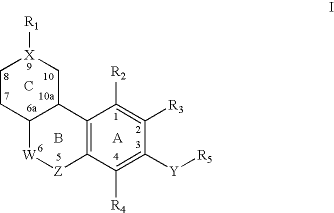
wherein:
the C ring contains one double bond;
W is selected from C═O, C═S or C═CH₂;
X is selected from C, CH, N, S, O, SO or SO₂;
Y is selected from O, S, C═C or C≡C;
Z is selected from 0, NH, N-alkyl where the alkyl group has 1 to about 5 carbon atoms or N-substituted alkyl, where the alkyl group has 1 to about 5 carbon atoms and is substituted with at least one substituent group in any possible position;
when X is S, O, SO or SO₂, R₁ is not present, or
when X is N, R₁ is selected from H, alkyl, alkoxy-alkyl, alkylmercapto, alkylamino, SO₃alkyl, SO₂NQ₁Q₂, CONQ₁Q₂, COC(halogen)₃ or alkyl substituted in any possible position with at least one member selected from OH, CHO, COOH, C(halogen)₃, N₃, NCS, CN, PO₃H₂, SO₃H, or SO₃alkyl, or
when X is C or CH, R₁ is selected from H, halogen, N₃, NCS, CN, NO₂, NQ₁Q₂, ═O, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, ═CH₂, alkyl, alcohol, alkoxy, alkylmercapto, alkylamino, di-alkylamino or alkyl substituted in any possible position with at least one substituent group,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, hydroxyloweralkyl or alkyl-NQ₁Q₂.
R₂ is selected from H, OH, OCH₃, OPO₃H₂, OSO₃H, PO₃H₂, SO₃H, halogen, NQ₁Q₂, COOQ₃, OQ₃, CQ₃, C(halogen)₃, alkyl-hydroxyl, NH—COalkyl, NH—COaryl, O-COalkyl, O—COalkyl-T₁, O—CO-T₁, NH—COalkyl-T₁, NH—CO-T₁, O-alkyl-T₁, O-T₁, NH-alkyl-T₁, NH-T1, SO₃alkyl or SO₂NQ₁Q₂,
T₁ is in any possible position and is selected from PO₃H, SO₃H, an alkyl group containing from 1 to about 16 carbons, tetrahydropyrrole, morpholine, thiomorpholine, piperazine, a heterocyclic ring or NQ₁Q₂ and T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, hydroxyloweralkyl or alkyl-NQ₁Q₂;
R₃ is selected from H, OH, halogen, C(halogen)₃, CN, N₃, NCS, NQ₁Q₂ or an alkyl group having 1 to about 4 carbon atoms,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members;
R₄ is selected from H, OH, halogen, CN, N₃, NCS, NQ₁Q₂ or an alkyl group having 1 to about 4 carbon atoms,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members; and
R₅ is selected from -D₁-D₂-T₂ or -D₂-T₂,
D₁, is optionally present and if present, is selected from an alkyl group, a carbocyclic ring, a heterocyclic ring, N-alkyl or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃ or adamantan-2-ylidenemethyl-T₃, alkylamino, di-alkylamino or NH
T₂ is selected from, in any possible position, a substituent group or —CO-T₄,
T₃ is an alkyl group having from 0 to about 9 carbon atoms,
T₄ is selected from H, C-(halogen)₃, OH, NH₂, alkylamino, di-alkylamino, NO₂, alkyl, alkoxy, a heterocyclic ring or a heteroaromatic ring.

2. The compound of claim 1 wherein X is C or CH and R₁ is selected from H, halogen, ═CH₂, an alkyl group having 1 to about 5 carbon atoms or an alkyl group having 1 to about 5 carbon atoms and substituted in any possible position with at least one member selected from OH, CHO, COOH, CH₂OH, halogen, C(halogen)₃, N₃, NCS, CN, PO₃H₂, SO₃H, or SO₃alkyl, SO₂NQ₁Q₂, CONQ₁Q₂ or NQ₁Q2.

3. The compound of claim 1 wherein:
D₁ is optionally present and if present, is selected from alkyl, a carbocyclic ring having 5 to 6 ring members, a heterocyclic ring having 5 to 6 ring members and 1,3 di-heteroatoms each independently selected from O, S, N and NH;
D₂ is selected from an alkyl group having from one to about sixteen carbon atoms, CH═CH, C≡C, a bicyclic ring, a tricyclic terpine, 1-adamantyl-T₃, 2-adamantyl-T₃, adamantan-1-ylmethyl-T₃ or adamantan-2-ylidenemethyl-T₃, alkylamino, di-alkylamino or NH; and
T₄ is selected from alkyl, a heterocyclic ring or a heteroaromatic ring.

4. The compound of claim 1 wherein W is C═O; Z is 0; X is C or CH; and R1 is selected from H, halogen, ═O, ═CH2, an alkyl group having between 1 to about 5 carbon atoms or a alkyl group having between 1 to about 5 carbon atoms and substituted in any possible position with at least one substituent selected from OH, CHO, COOH, CH₂OH, halogen, C(halogen)₃, N₃, NCS, CN, PO₃H₂, SO₃H, SO₃Alkyl, SO₂NQ₁Q₂, CONQ₁Q₂, and NQ₁Q₂, where
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members.

5. The compound of claim 1 wherein D₁ is, if present, selected from a carbocyclic ring having 5 to 6 ring members, a heterocyclic ring having 5 to 6 ring members and 1,3 di-heteroatoms each independently selected from O, S, N and NH,
D₂ is selected from an alkyl group having from one to about sixteen carbon atoms, CH═CH, C≡C, a bicyclic ring, a tricyclic ring, 1-adamantyl-T₃, 2-adamantyl-T₃, adamantan-1-ylmethyl-T₃ or adamantan-2-ylidenemethyl-T₃, alkylamino, di-alkylamino or NH, and
T₄ is selected from alkyl, a heterocyclic ring or a heteroaromatic ring.

6. The compound of claim 1 wherein:
the C ring double bond is in the 6a-10a position;
W is C═O;
Z is O;
X is CH;
R₁ is selected from OH, CH₂OH; halogen and C(halogen)₃;
R₂ is selected from H, OH, OCH₃, OPO₃H₂, OSO₃H, PO₃H₂, SO₃H, halogen, NQ₁Q₂, COOQ₃, OQ₃, NH—COalkyl, NH—CO-aryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂,
Q₃ is selected from H, alkyl, alcohol or alkyl-NQ₁Q₂;
R₃ is selected from H, OH, halogen, CN, N₃, NCS, NQ₁Q₂ or an alkyl group having 1 to about 4 carbon atoms,
R₄ is selected from H, OH, halogen, CN, N₃, NCS, NQ₁Q₂ or an alkyl group having 1 to about 4 carbon atoms; and
R₅ is -D₁-D₂-T₂ or -D₂-T₂,
D₁, if present, is selected from alkyl, a carbocyclic ring, a heterocyclic ring, alkylamino or NH,
D₂ is selected from an alkyl group having from one to about sixteen carbon atoms, CH═CH, C≡C, a bicyclic ring, a tricyclic ring, 1-adamantyl-T₃, 2-adamantyl-T₃, adamantan-1-ylmethyl-T₃, or adamantan-2-ylidenemethyl-T₃, alkylamino, di-alkylamino or NH.

7. The compound of claim 1 wherein:
the C ring double bond is in the 6a-10a position;
W is C═O;
Z is O;
X is N and R₁ is CH₂OH, or
X is C and R₁ is selected from OH, CH₂OH, halogen or C(halogen)₃;
R₂ is selected from H, OH, OCH₃, OPO₃H₂, OSO₃H, PO₃H₂, SO₃H, halogen, NQ₁Q₂, COOQ₃, OQ₃, NH—COalkyl, NH—CO-aryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl or SO₂NQ₁Q₂; and
R₅ is -D₁-D₂-T₂ or -D₂-T₂,
D₁, if present, is selected from an alkyl, a carbocyclic ring having 4 to 6 ring members or a heterocyclic ring having 4 to 6 ring members and 1,3 di-heteroatoms each heteroatom independently selected from O, S and N,
D₂ is selected from an alkyl group having from one to about sixteen carbon atoms, CH═CH, C≡C, alkylamino, d-alkylamino, NH, a bicyclic ring, a tricyclic ring, 1-adamantyl-T₃, 2-adamantyl-T₃, adamantan-1-ylmethyl-T₃ or adamantan-2-ylidenemethyl-T₃,
T₄ is selected from alkyl, C(halogen)₃ aminoalkyl, di-aminoalkyl, NH2, a heterocyclic ring or a heteroaromatic ring.

8. The compound of claim 1 wherein:
the C ring double bond is in the 6a-10a position;
W is C═O;
X is selected from C or N;
Y is selected from O, S, C═C or C≡C,
Z is O;
when X is C, R₁ is selected from OH or CH₂OH, when X is N, R₁ is selected from CH₂OH;
R₂ is selected from H, OH, OCH₃, OPO₃H₂, OSO₃H, PO₃H₂, SO₃H, halogen, C(halogen)₃, alcohol, NQ₁Q₂, COOQ₃, OQ₃, NH—COalkyl, NH—CO-aryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl or SO₂NQ₁Q₂; and
R₅ is selected from -D₁-D₂-T₂ or -D₂-T₂,
D₂ is selected from an alkyl group having from one to about sixteen carbon atoms, CH═CH, C≡C, a bicyclic ring, a tricyclic ring, 1-adamantyl-T₃, 2-adamantyl-T₃, adamantan-1-ylmethyl-T₃, or adamantan-2-ylidenemethyl-T₃, alkylamino, di-alkylamino or NH.

9. The compound of claim 1 wherein:
R₅ is selected from -D₁-D₂-T₂ or -D₂-T₂;
D₁ is optionally present and if present, is selected from alkyl, a carbocyclic ring having 4 to 6 ring members or a heterocyclic ring having 4 to 6 ring members and 1,3 di-heteroatoms each heteroatom independently selected from O, S and N; and
T₄ is selected from alkyl, C(halogen)₃ aminoalkyl, di-aminoalkyl, NH₂, a heterocyclic ring or a heteroaromatic ring.

10. A compound of formula II, and physiologically acceptable salts thereof, 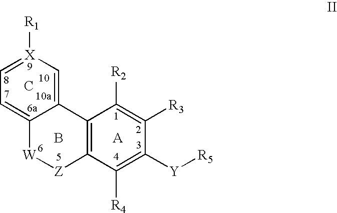
wherein:
W is selected from C═O, C═S, or C═CH₂;
X is selected from C, CH or N;
Y is selected from O, S, C═C or C≡C;
Z is selected from O, NH, N-alkyl where the alkyl group has 1 to about 5 carbon atoms or N-substituted alkyl, where the alkyl group has 1 to about 5 carbon atoms and is substituted with at least one substituent group in any possible position;
R₁ is selected from H, halogen, N₃, NCS, CN, NO₂, NQ₁Q₂, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, alkyl, alkyl substituted in any possible position with at least one substituent group,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, alcohol, or alkyl-NQ₁Q₂;
R₂ is selected from H, OH, OCH₃, OPO₃H₂, OSO₃H, PO₃H₂, SO₃H, halogen, C(halogen)₃, alcohol, NQ₁Q₂, COOQ₃, OQ₃, alkyl-hydroxyl, NH—COalkyl, NH—COaryl, O—COalkyl, O—COalkyl-T₁, O—CO-T₁, SO₂NQ₁Q₂, CONQ₁Q₂, NH—COalkyl-T₁, NH—CO-T₁, O-alkyl-T₁, O-T₁, NH-alkyl-T₁, NH-T₁, SO₃alkyl or SO₂NQ₁Q₂,
T₁ is in any possible position and is selected from PO₃H, SO₃H, an alkyl group containing from 1 to about 16 carbon atoms, tetrahydropyrrole, morpholine, thiomorpholine, piperazine, a heterocyclic ring or NQ₁Q₂, and T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, alcohol, or alkyl-NQ₁Q₂;
R₃ is selected from H, OH, halogen, C(halogen)₃, CN, N₃, NCS, NQ₁Q₂ or C1 to C4 alkyl,
Q₁ and Q₂ are each independently is selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members;
R₄ is selected from H, OH, halogen, C(halogen)₃, CN, N₃, NCS, NQ₁Q₂ or C1 to C4 alkyl;
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members; and
R₅ is selected from -D₁-D₂-T₂ or -D₂-T₂,
D₁ is optionally present and if present, is selected from alkyl, a carbocyclic ring, a heterocyclic ring, alkylamino, di-alkylamino or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃, or adamantan-2-ylidenemethyl-T₃, alkylamino, di-alkylamino or NH,
T₂ is selected from, in any possible position, a substituent group or —CO-T₄,
T₃ is selected from an alkyl group having from 0 to about 9 carbon atoms,
T₄ is selected from H, C(halogen)₃, OH, NH₂, NO₂, alkyl, alkoxy, a heterocyclic ring or a heteroaromatic ring.

11. The compound of claim 10 wherein R₁ is selected from H, halogen, OH, an alkyl group having 1 to about 5 carbon atoms or an alkyl group having 1 to about 5 carbon atoms and substituted in any possible position with at least one member selected from OH, CHO, COOH, C(halogen)₃, N₃, NCS, CN, PO₃H₂, SO₃H, or SO₃alkyl.

12. The compound of claim 10 wherein:
R₅ is selected from -D₁-D₂-T₂ or -D₂-T2
D₁ is selected from alkylamino, di-alkylamino, NH, a carbocyclic ring having 4 to 6 ring members or a heterocyclic ring having 4 to 6 ring members and 1,3 di-heteroatoms each heteroatom independently selected from O, S and N,
D₂ is selected from an alkyl group having from one to about sixteen carbon atoms, CH═CH, C≡C, a bicyclic ring, a tricyclic terpine, 1-adamantyl-T₃, 2-adamantyl-T₃, adamantan-1-ylmethyl-T₃ or adamantan-2-ylidenemethyl-T₃, alkylamino, di-alkylamino or NH, and
T₄ comprises alkyl, a heterocyclic ring or a heteroaromatic ring.

13. The compound of claim 10 wherein:
W is C═O;
X is selected from C or N;
Y is selected from O, S, C═C, C≡C;
Z is O;
R₁ is selected from methyl, OH or CH₂OH;
R₂ is selected from H, OH, OCH₃, OPO₃H₂, OSO₃H, PO₃H₂, SO₃H, halogen, C(halogen)₃, alcohol, NQ₁Q₂, COOQ₃, OQ₃, NH—COalkyl, NH—CO-aryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂; and
R₅ is selected from -D₁-D₂-T₂ or -D₂-T₂,
D₁ is optionally present and if present, is selected from alkyl, a carbocyclic ring, a heterocyclic ring, alkylamino or NH,
D₂ is selected from an alkyl group having from one to about sixteen carbon atoms, CH═CH, C≡C, a bicyclic ring, a tricyclic ring, 1-adamantyl-T₃, 2-adamantyl-T₃, adamantan-1-ylmethyl-T₃, or adamantan-2-ylidenemethyl-T₃, alkylamino, di-alkylamino or NH.

14. The compound of claim 10 wherein:
R₅ is selected from -D₁-D₂-T₂ or -D₂-T₂,
D₁ is optionally present and if present, is selected from alkyl, a carbocyclic ring having 4 to 6 ring members or a heterocyclic ring having 4 to 6 ring members and 1,3 di-heteroatoms each heteroatom independently selected from O, S and N,
D₂ is selected from an alkyl group having from one to about sixteen carbon atoms, CH═CH, C≡C, alkylamino, di-alkylamino, NH, a bicyclic ring, a tricyclic ring, 1-adamantyl-T₃, 2-adamantyl-T₃, adamantan-1-ylmethyl-T₃ or adamantan-2-ylidenemethyl-T₃, and
T₄ is selected from alkyl, C(halogen)₃ NH₂, a heterocyclic ring or a heteroaromatic ring.

15. The compound of claim 10, wherein:
Y is O;
X is selected from C, CH, and N;
R₁ is selected from H, halogen, N₃, NCS, CN, NO₂, NQ₁Q₂, ═O, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, ═CH₂, alkyl, alcohol, alkoxy, alkylmercapto, alkylamino, di-alkylamino or alkyl substituted in any possible position with at least one substituent group,
where Q₁ and Q₂ are each independently selected from H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members,
Q₃ comprises H, alkyl, alcohol, or alkyl-NQ₁Q₂;
R₂ is selected from H, OH, OCH₃, OPO₃H₂, OSO₃H, PO₃H₂, SO₃H, halogen, NQ₁Q₂, COOQ₃, OQ₃, CQ₃, C(halogen)₃, alcohol, NH—COalkyl, NH—COaryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂,
T₁ is in any possible position and comprises PO₃H, SO₃H, an alkyl group containing from 1 to about 16 carbons, tetrahydropyrrole, morpholine, thiomorpholine, piperazine, a heterocyclic ring or NQ₁Q₂,
T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members,
Q₃ is selected from H, alkyl, alcohol, or alkyl-NQ₁Q₂;
R₃ is selected from H, OH, halogen, C(halogen)₃, CN, N₃, NCS, NQ₁Q₂ or an alkyl group having 1 to about 4 carbon atoms,
Q₁ and Q₂ are each independently selected from H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members;
R₄ is selected from H, OH, halogen, CN, N₃, NCS, NQ₁Q₂ or an alkyl group having 1 to about 4 carbon atoms,
Q₁ and Q₂ are each independently selected from H or alkyl, or
Q₁ and Q₂ together comprise part of a heterocyclic ring having about 4 to about 7 ring members and optionally one additional heteroatom selected from O, N or S, or
Q₁ and Q₂ together comprise part of an imide ring having about 5 to about 6 members; and
R₅ is -D₁-D₂-T₂ or -D₂-T₂,
D₁ if present, is selected from an alkyl group, a carbocyclic ring, a heterocyclic ring, N-alkyl or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃ or adamantan-2-ylidenemethyl-T₃, alkylamino, di-alkylamino or NH,
T₂ is, in any possible position, selected from a substituent group or —CO-T₄,
T₃ is an alkyl group having from 0 to about 9 carbon atoms,
T₄ is selected from H, C(halogen)₃, OH, NH₂, alkylamino, di-alkylamino, NO₂, alkyl, alkoxy, a heterocyclic ring or a heteroaromatic ring.

16. The compound of claim 10 wherein W is C═O.

17. The compound of claim 10 wherein:
W is C═O;
X is C or N;
Z is O;
R₁ is selected from methyl, OH, CH₂OH; halogen or C(halogen)₃;
R₂ is selected from H, OH, OCH₃, OPO₃H₂, OSO₃H, PO₃H₂, SO₃H, halogen, C(halogen)₃, NQ₁Q₂, COOQ₃, OQ₃, NH—COalkyl, NH—CO-aryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl, SO₂NQ₁Q₂ or CONQ₁Q₂; and
R₅ is -D₁-D₂-T₂ or -D₂-T₂,
D₁, if present, is selected from a carbocyclic ring, a heterocyclic ring, alkylamino or NH,
D₂ is selected from an alkyl group having from one to about sixteen carbon atoms, CH═CH, C≡C, a bicyclic ring, a tricyclic ring, 1-adamantyl-T₃, 2-adamantyl-T₃, adamantan-1-ylmethyl-T₃, or adamantan-2-ylidenemethyl-T₃, alkylamino, di-alkylamino or NH,
T₂ is, in any possible position, selected from a substituent group or —CO-T₄,
T₃ is an alkyl group having from 0 to about 9 carbon atoms,
T₄ is selected from H, C(halogen)₃, OH, NH₂, NO₂, alkyl, alkoxy, alkylamino, di-alkylamino, a heterocyclic ring or a heteroaromatic ring.

18. A method of using a cannabinoid compound as a fluorophore to generate a fluorescence emission signal comprising the steps of:
providing a tricyclic cannabinoid compound having a excitation range and an emission range, the tricyclic cannabinoid compound having the following structure: 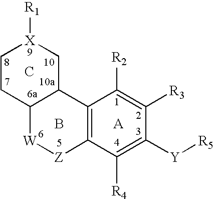
wherein:
the C ring contains one double bond or three double bonds;
W is selected from C═O, C═S or C═CH₂;
if the C ring contains one double bond X is selected from C, CH, N, S, O, SO or SO₂ and if the C ring contains three double bonds X is selected from C, CH or N;
Y is selected from O, S, C═C or C≡C
Z is selected from O, NH, N-alkyl where the alkyl group has 1 to about 5 carbon atoms or N-substituted alkyl, where the alkyl group has 1 to about 5 carbon atoms and is substituted with at least one substituent group in any possible position;
R₁, R₂, R₃, R₄ and R₅ are not limited; exciting the compound with electromagnetic radiation; and detecting electromagnetic radiation fluorescently emitted by the compound.

19. The method of claim 18 wherein the tricyclic cannabinoid compound fluorescently emits electromagnetic radiation in the ultraviolet-visible wavelength ranges.

20. The method of claim 18 wherein the tricyclic cannabinoid compound fluorescently emits electromagnetic radiation in the range of about 390 nm to about 550 nm.

21. The method of claim 18 wherein W is C═O.

22. The method of claim 18 wherein the tricyclic cannabinoid compound has the following structural formula, and physiologically acceptable salts thereof, 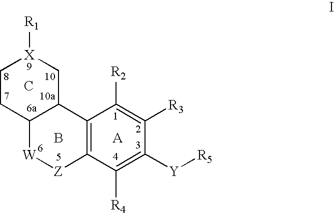
wherein:
the C ring contains one double bond;
W is selected from C═O, C═S or C═CH₂;
X is selected from C, CH, N, S, O, SO or SO₂;
Y is selected from O, S, C═C or C≡C;
Z is selected from O, NH, N-alkyl where the alkyl group has 1 to about 5 carbon atoms or N-substituted alkyl, where the alkyl group has 1 to about 5 carbon atoms and is substituted with at least one substituent group in any possible position;
when X is S, O, SO or SO₂, R₁ is not present, or
when X is N, R₁ is selected from H, alkyl, alkoxy-alkyl, alkylmercapto, alkylamino, SO₃alkyl, SO₂NQ₁Q₂, CONQ₁Q₂, COC(halogen)₃ or alkyl substituted in any possible position with at least one member selected from OH, CHO, COOH, C(halogen)₃, N₃, NCS, CN, PO₃H₂, SO₃H, or SO₃alkyl, or
when X is C or CH, R₁ is selected from H, halogen, N₃, NCS, CN, NO₂, NQ₁Q₂, ═O, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, ═CH₂, alkyl, alcohol, alkoxy, alkylmercapto, alkylamino, di-alkylamino or alkyl substituted in any possible position with at least one substituent group,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, hydroxyloweralkyl or alkyl-NQ₁Q₂,
R₂ is selected from H, OH, OCH₃, OPO₃H₂, OSO₃H, PO₃H₂, SO₃H, halogen, NQ₁Q₂, COOQ₃, OQ₃, CQ₃, C(halogen)₃, alkyl-hydroxyl, NH—COalkyl, NH—COaryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl or SO₂NQ₁Q₂,
T₁ is in any possible position and is selected from PO₃H, SO₃H, an alkyl group containing from 1 to about 16 carbons, tetrahydropyrrole, morpholine, thiomorpholine, piperazine, a heterocyclic ring or NQ₁Q₂ and T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, hydroxyloweralkyl or alkyl-NQ₁Q₂;
R₃ is selected from H, OH, halogen, C(halogen)₃, CN, N₃, NCS, NQ₁Q₂ or an alkyl group having 1 to about 4 carbon atoms,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members;
R₄ is selected from H, OH, halogen, CN, N₃, NCS, NQ₁Q₂ or an alkyl group having 1 to about 4 carbon atoms,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members; and
R₅ is selected from -D₁-D₂-T₂ or -D₂-T₂,
D₁, is optionally present and if present, is selected from an alkyl group, a carbocyclic ring, a heterocyclic ring, N-alkyl or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃ or adamantan-2-ylidenemethyl-T₃, alkylamino, di-alkylamino or NH
T₂ is selected from, in any possible position, a substituent group or —CO-T₄,
T₃ is an alkyl group having from 0 to about 9 carbon atoms,
T₄ is selected from H, C-(halogen)₃, OH, NH₂, alkylamino, di-alkylamino, NO₂, alkyl, alkoxy, a heterocyclic ring or a heteroaromatic ring.

23. The method of claim 18 wherein the tricyclic cannabinoid compound has the following structural formula, and physiologically acceptable salts thereof, 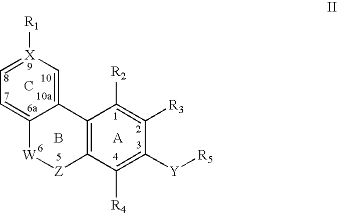
wherein:
W is selected from C═O, C═S, or C═CH₂;
X is selected from C, CH or N;
Y is selected from O, S, C═C or C≡C;
Z is selected from 0, NH, N-alkyl where the alkyl group has 1 to about 5 carbon atoms or N-substituted alkyl, where the alkyl group has 1 to about 5 carbon atoms and is substituted with at least one substituent group in any possible position;
R₁ is selected from H, halogen, N₃, NCS, CN, NO₂, NQ₁Q₂, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, alkyl, alkyl substituted in any possible position with at least one substituent group,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, alcohol, or alkyl-NQ₁Q₂;
R₂ is selected from H, OH, OCH₃, OPO₃H₂, OSO₃H, PO₃H₂, SO₃H, halogen, C(halogen)₃, alcohol, NQ₁Q₂, COOQ₃, OQ₃, alkyl-hydroxyl, NH—COalkyl, NH—COaryl, O—COalkyl, O—COalkyl-T₁, O—CO-T₁, SO₂NQ₁Q₂, CONQ₁Q₂, NH—COalkyl-T₁, NH—CO-T₁, O-alkyl-T₁, O-T₁, NH-alkyl-T₁, NH-T₁, SO₃alkyl or SO₂NQ₁Q₂,
T₁ is in any possible position and is selected from PO₃H, SO₃H, an alkyl group containing from 1 to about 16 carbon atoms, tetrahydropyrrole, morpholine, thiomorpholine, piperazine, a heterocyclic ring or NQ₁Q₂, and T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, alcohol, or alkyl-NQ₁Q₂;
R₃ is selected from H, OH, halogen, C(halogen)₃, CN, N₃, NCS, NQ₁Q₂ or C1 to C4 alkyl,
Q₁ and Q₂ are each independently is selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members;
R₄ is selected from H, OH, halogen, C(halogen)₃, CN, N₃, NCS, NQ₁Q₂ or C1 to C4 alkyl;
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members; and
R₅ is selected from -D₁-D₂-T₂ or -D₂-T2,
D₁ is optionally present and if present, is selected from alkyl, a carbocyclic ring, a heterocyclic ring, alkylamino, di-alkylamino or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan1-ylmethyl-T₃, or adamantan-2-ylidenemethyl-T₃, alkylamino, di-alkylamino or NH,
T₂ is selected from, in any possible position, a substituent group or —CO-T₄,
T₃ is selected from an alkyl group having from 0 to about 9 carbon atoms,
T₄ is selected from H, C(halogen)₃, OH, NH₂, NO₂, alkyl, alkoxy, a heterocyclic ring or a heteroaromatic ring.

24. A method of preferentially stimulating one of the CB1 or CB2 receptors in an individual or animal, comprising administering to the individual or animal a pharmacological composition comprising a therapeutically effective amount of the following compound, and physiologically acceptable salts thereof, 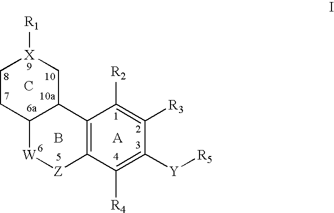
wherein:
the C ring contains one double bond;
W is selected from C═O, C═S or C═CH₂;
X is selected from C, CH, N, S, O, SO or SO₂;
Y is selected from O, S, C═C or C≡C;
Z is selected from O, NH, N-alkyl where the alkyl group has 1 to about 5 carbon atoms or N-substituted alkyl, where the alkyl group has 1 to about 5 carbon atoms and is substituted with at least one substituent group in any possible position;
when X is S, O, SO or SO₂, R₁ is not present, or
when X is N, R₁ is selected from H, alkyl, alkoxy-alkyl, alkylmercapto, alkylamino, SO₃alkyl, SO₂NQ₁Q₂, CONQ₁Q₂, COC(halogen)₃ or alkyl substituted in any possible position with at least one member selected from OH, CHO, COOH, C(halogen)₃, N₃, NCS, CN, PO₃H₂, SO₃H, or SO₃alkyl, or
when X is C or CH, R₁ is selected from H, halogen, N₃, NCS, CN, NO₂, NQ₁Q₂, ═O, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, ═CH₂, alkyl, alcohol, alkoxy, alkylmercapto, alkylamino, di-alkylamino or alkyl substituted in any possible position with at least one substituent group,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, hydroxyloweralkyl or alkyl-NQ₁Q₂,
R₂ is selected from H, OH, OCH₃, OPO₃H₂, OSO₃H, PO₃H₂, SO₃H, halogen, NQ₁Q₂, COOQ₃, OQ₃, CQ₃, C(halogen)₃, alkyl-hydroxyl, NH—COalkyl, NH—COaryl, O—COalkyl, O—COalkyl-T₁, O—CO-T₁, NH—COalkyl-T₁, NH—CO-T₁, O-alkyl-T₁, O-T₁, NH-alkyl-T₁, NH-T₁, SO₃alkyl or SO₂NQ₁Q₂,
T₁ is in any possible position and is selected from PO₃H, SO₃H, an alkyl group containing from 1 to about 16 carbons, tetrahydropyrrole, morpholine, thiomorpholine, piperazine, a heterocyclic ring or NQ₁Q₂ and T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, hydroxyloweralkyl or alkyl-NQ₁Q₂;
R₃ is selected from H, OH, halogen, C(halogen)₃, CN, N₃, NCS, NQ₁Q₂ or an alkyl group having 1 to about 4 carbon atoms,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members;
R₄ is selected from H, OH, halogen, CN, N₃, NCS, NQ₁Q₂ or an alkyl group having 1 to about 4 carbon atoms,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members; and
R₅ is selected from -D₁-D₂-T₂ or -D₂-T₂,
D₁, is optionally present and if present, is selected from an alkyl group, a carbocyclic ring, a heterocyclic ring, N-alkyl or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃ or adamantan-2-ylidenemethyl-T₃, alkylamino, di-alkylamino or NH
T₂ is selected from, in any possible position, a substituent group or —CO-T₄,
T₃ is an alkyl group having from 0 to about 9 carbon atoms,
T₄ is selected from H, C-(halogen)₃, OH, NH₂, alkylamino, di-alkylamino, NO₂, alkyl, alkoxy, a heterocyclic ring or a heteroaromatic ring.

25. The method of claim 24 wherein the CB2 receptor is preferentially stimulated.

26. The method of claim 24 wherein the compound is purified.

27. A method of preferentially stimulating one of the CB1 or CB2 receptors in an individual or animal, comprising administering to the individual or animal a pharmacological composition comprising a therapeutically effective amount of the following compound, and physiologically acceptable salts thereof, 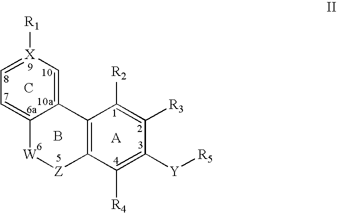
wherein:
W is selected from C═O, C═S or C═CH₂;
X is selected from C, CH or N;
Y is selected from O, S, C═C or C≡C;
Z is selected from O, NH, N-alkyl where the alkyl group has 1 to about 5 carbon atoms or N-substituted alkyl, where the alkyl group has 1 to about 5 carbon atoms and is substituted with at least one substituent group in any possible position;
R₁ is selected from H, halogen, N₃, NCS, CN, NO₂, NQ₁Q₂, OQ₃, OAc, O-acyl, O-aroyl, NH-acyl, NH-aroyl, CHO, C(halogen)₃, COOQ₃, PO₃H₂, SO₃H, SO₃alkyl, SO₂NQ₁Q₂, CONQ₁Q₂, COC(halogen)₃, alkyl, alkyl substituted in any possible position with at least one substituent group,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, alcohol, or alkyl-NQ₁Q₂;
R₂ is selected from H, OH, OCH₃, OPO₃H₂, OSO₃H, PO₃H₂, SO₃H, halogen, C(halogen)₃, alcohol, NQ₁Q₂, COOQ₃, OQ₃, alkyl-hydroxyl, NH—COalkyl, NH—COaryl, O—COalkyl, O—COalkyl-T₁, O—CO-T₁, SO₂NQ₁Q₂, CONQ₁Q₂, NH—COalkyl-T₁, NH—CO-T₁, O-alkyl-T₁, O-T₁, NH-alkyl-T₁, NH-T₁, SO₃alkyl or SO₂NQ₁Q₂,
T₁ is in any possible position and is selected from PO₃H, SO₃H, an alkyl group containing from 1 to about 16 carbon atoms, tetrahydropyrrole, morpholine, thiomorpholine, piperazine, a heterocyclic ring or NQ₁Q₂, and T₁ may be substituted in any possible position with at least one member selected from a substituent group, OPO₃H₂, OSO₃H, PO₃H₂, a heterocyclic ring or a heteroaromatic ring,
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members,
Q₃ is selected from H, alkyl, alcohol, or alkyl-NQ₁Q₂;
R₃ is selected from H, OH, halogen, C(halogen)₃, CN, N₃, NCS, NQ₁Q₂ or C1 to C4 alkyl,
Q₁ and Q₂ are each independently is selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members;
R₄ is selected from H, OH, halogen, C(halogen)₃, CN, N₃, NCS, NQ₁Q₂ or C1 to C4 alkyl;
Q₁ and Q₂ are each independently selected from H or alkyl, or
Q₁ and Q₂ together are part of a heterocyclic ring having about 4 to about 7 ring members and optionally one additional heteroatom selected from O, N or S, or
Q₁ and Q₂ together are part of an imide ring having about 5 to about 6 members; and
R₅ is selected from -D₁-D₂-T₂ or -D₂-T₂
D₁ is optionally present and if present, is selected from alkyl, a carbocyclic ring, a heterocyclic ring, alkylamino, di-alkylamino or NH,
D₂ is selected from an alkyl group having from one to about sixteen carbon atoms, CH═CH, C≡C, a bicyclic ring, a tricyclic ring, a heterocyclic ring, an aromatic ring, a heteroaromatic ring, 1-adamantyl-T₃, 2-adamantyl-T₃, adamantan-1-ylmethyl-T₃, or adamantan-2-ylidenemethyl-T₃, alkylamino, di-alkylamino or NH,
T₂ is selected from, in any possible position, a substituent group or —CO-T₄,
T₃ is selected from an alkyl group having from 0 to about 9 carbon atoms,
T₄ is selected from H, C(halogen)₃, OH, NH₂, NO₂, alkyl, alkoxy, a heterocyclic ring or a heteroaromatic ring.

28. The method of claim 27 wherein the CB2 receptor is preferentially stimulated.

29. The method of claim 27 wherein the compound is purified.
